(19) United States
(12) Patent Application Publication
(10) Pub. No.: US 2020/0131251 A1
(43) Pub. Date: Apr. 30, 2020

(54) IMPROVED METHODS FOR ENHANCING ANTIBODY PRODUCTIVITY IN MAMMALIAN CELL CULTURE AND MINIMIZING AGGREGATION DURING DOWNSTREAM, FORMULATION PROCESSES AND STABLE ANTIBODY FORMULATIONS OBTAINED THEREOF

(71) Applicant: SERUM INSTITUTE OF INDIA PRIVATE LIMITED, Maharashtra (IN)

(72) Inventors: Dhere Rajeev MHALASAKANT, Maharashtra (IN); Pisal Sambhaji SHANKAR, Maharashtra (IN); Peddi Reddy Srinivas REDDY, Maharashtra (IN); Singh Digamber CHAHAR, Maharashtra (IN); Yeolekar Leena RAVINDRA, Pune (IN); Chouhan Pankaj SINGH, Maharashtra (IN); Avalaskar Nikhil DATTATRAY, Maharashtra (IN)

(21) Appl. No.: 16/472,673

(22) PCT Filed: Dec. 20, 2017
(86) PCT No.: PCT/IB2017/058194

§ 371 Date: Jun. 21, 2019

(30) Foreign Application Priority Data
Dec. 23, 2016 (IN) 2016621044139

(51) Int. Cl. C07K 16/10; C12N 5/078; A61K 39/395; A61K 9/19

(52) U.S. Cl. CPC C07K 16/1081; C12N 5/0634; C07K 16/10; C07K 2317/92; A61K 39/39525; A61K 9/19; C07K 2317/14; A61K 39/39591

(57) ABSTRACT

The invention describes an efficient platform for antibody manufacturing and formulation that provides i) cell culture process with improved feeding strategy resulting in high antibody titer between 2 gm/L to 5 gm/L; ii) improved purification process showing optimal percentage recovery, high purity monomer content, minimum aggregation/particulate formation, minimum impurity levels; and iii) high concentration stable liquid formulation with optimal osmolality and low viscosity across different temperature excursions and devoid of aggregation. The preferred antibodies include IgG1 monoclonal antibody specific to the Dengue virus epitope in domain III of the E protein and IgG1 monoclonal antibody specific to the rabies virus surface G glycoprotein.

### BACKGROUND OF THE INVENTION

Upstream, Downstream and formulation development can often be the rate-limiting step in the early introduction of biopharmaceuticals into clinical trials. For instance, Dengue is the most important mosquito-borne viral disease affecting humans. Half of the world population lives in areas at risk for dengue, resulting in an estimated 390 million infections per year globally. Currently no antiviral agents are approved for treating dengue and recent vaccine trials have fallen short of expectations. The leading vaccine candidate recently demonstrated limited efficacy, estimated to be between 30%-60%, with limited to no significant protection against DENV-2. Recently a non-immunodominant, but functionally relevant, epitope in domain III of the E protein has been identified, and subsequently an engineered antibody, Ab513 is being developed that exhibits high-affinity binding to, and broadly neutralizes, multiple genotypes within all four serotypes (Refer Ram Sasisekharan et al Cell 162, 1-12, Jul. 30, 2015; Samir Bhatt et al, Nature, 2013 Apr. 25; 496 7446: 504-507). Thus if we consider global medical demand for a Dengue monoclonal antibody, recent estimates indicate that up to 390 million dengue infections occur every year globally with >90 million presenting with disease making DENV a major global threat. If we assume that 30% of the 16 million diagnosed dengue cases go to hospital then about 5 million will require said Dengue monoclonal antibody, which implies that “purified antibody” above 4 gm/L becomes a prerequisite to meet global demand of such a life saving antibody. Further, Dengue disease burden is high in developing countries where availability of electrical power and refrigeration are often inadequate and therefore antibody stability across temperature excursions assumes greater relevance for these regions.

Indeed, if it was possible to have a platform process that could be employed for manufacturing and formulating all monoclonal antibody (mAb) candidates it would greatly reduce the time and resources needed for process development. This can have a significant impact on the number of clinical candidates that can be introduced into clinical trials. Also, processes developed for early stage clinical trials, including those developed using a platform, may be non-optimal with respect to process economics, yield, pool volumes, throughput and may not be suitable for producing the quantities required for late stage or commercial campaigns. Another important consideration is the speed of process development given that process development needs to occur prior to introduction of a therapeutic candidate into clinical trials. (Refer Abhinav A. Shukla et al Journal of Chromatography B, 848 (2007) 28-39).

Typically mammalian cell culture media is based on commercially available media formulations, including, for example, DMEM or Ham's F12. Often media formulations are not sufficiently enriched to support increases in both cell growth and biologic protein expression. There remains a need for improved cell culture media, supplements, and cell culture methods for improved protein production. Increases in cell culture antibody titers to >2 g/L have been reported earlier. Refer F. Wurm, Nat. Biotechnol. 22 (2004) 1393. Further, in perfusion reactors, cells can reach much higher cell densities than in conventional batch or fed-batch reactors. (Refer Sven Sommerfeld et al Chemical Engineering and Processing 44 (2005) 1123-1137). However perfusion based processes are complex, costly and may also result in sterility issues and undesired heterogeneity in glycosylation pattern. Addition of animal-component-free hydrolysates (Bacto TC Yeastolate, Phytone Peptone) to chemically defined media is a common approach to increase cell density, culture viability and productivity in a timely manner. Hydrolysates are protein digests composed of amino acids, small peptides, carbohydrates, vitamins and minerals that provide nutrient supplements to the media. Non-animal derived hydrolysates from soy, wheat and yeast are used commonly in cell culture media and feeds to improve antibody titer (Refer U.S. Pat. No. 9,284,371). However, because of its composition complexity, lot-to-lot variations, undesirable attribute of making culture viscous, Yeast extract and hydrolysates can be a significant source of medium variability. Due to the complexity of antibody products that include isoforms and micro-heterogeneities, the performance of the cell culture process can have significant effects on product quality and potency, especially with respect to glycosylation, post-transcriptional modifications and impurity profiles.

At higher concentrations, proteins, particularly antibodies often exhibit characteristic problems including aggregation, precipitation, gelation, lowered stability, and/or increased viscosity.

Antibodies are recognized as possessing characteristics that tend to form aggregates and particulates in solution as they undergo degradation or aggregation or denaturation or chemical modifications resulting in the loss of biological activity during the manufacturing process and/or during storage with time. Antibody aggregates could be formed during cell culture expression, downstream purification, formulation and on storage. Cell culture harvest usually contains the highest level of aggregate in the process (Refer Deqiang Yu Journal of Chromatography A, 1457 (2016) 66-75). Degradation pathways for proteins can involve chemical instability (e.g., any process which involves modification of the protein by bond formation or cleavage resulting in a new chemical entity) or physical instability (e.g., changes in the higher order structure of the protein). The three most common protein degradation pathways are protein aggregation, deamidation and oxidation. Cleland et al Critical Reviews in Therapeutic Drug Carrier Systems 10(4): 307-377 (1993). Further, proteins also are sensitive to, for example, pH, ionic strength, thermal stress, shear and interfacial stresses, all of which can lead to aggregation and result in instability. For a protein to remain biologically active, a formulation must therefore preserve intact the conformational integrity of at least a core sequence of the protein's amino acids while at the same time protecting the protein's multiple functional groups from degradation.

A major problem caused by the aggregate formation is that during the administration the formulation may block syringes or pumps and rendering it unsafe to patients. Such protein modifications can also make them immunogenic resulting in the generation of anti-drug antibodies by the patient which can reduce the drug availability during subsequent injections or worse induce an autoimmune reaction. A major aim in the development of antibody formulations is to maintain protein solubility, stability and bioactivity.

Early suggestions about how to solve the problems of instability of protein therapeutics formulations included the lyophilization of the drug product, followed by reconstitution immediately or shortly prior to administration. However, Lyophilized formulations of antibodies have a number of limitations, including a prolonged process for lyophilization and resulting high cost for manufacturing. In addition, a lyophilized formulation has to be reconstituted aseptically and accurately by healthcare practitioners prior to administering to patients. The reconstitution step itself requires certain specific procedures, i.e. (1) a sterile diluent (i.e., water for intravenous administration and 5% dextrose in water for intramuscular administration) is added to the vial containing lyophilized antibody, slowly and aseptically, and the vial must be swirled very gently for 30 seconds to avoid foaming; (2) the reconstituted antibody may need to stand at room temperature for a minimum of 20 minutes until the solution clarifies; and (3) the reconstituted preparation must be administered within six (6) hours after the reconstitution. Such reconstitution procedure is cumbersome and the time limitation after the reconstitution can cause a great inconvenience in administering the formulation to patients, leading to significant waste, if not reconstituted properly, or if the reconstituted dose is not used within six (6) hours and must be discarded. Therefore, a liquid formulation is desirable due to factors of clinical and patient convenience as well as ease of manufacture. However liquid pharmaceutical formulations of protein therapeutics, i.e. antibodies should be long-term stable, contain a safe and effective amount of the pharmaceutical compound.

Removal of aggregates is more difficult than removal of process related impurities due to the biophysical similarities between the aggregate and monomer, the multiple sources and types of aggregate, and less understanding of aggregation mechanism.

One of the more recent challenges encountered during formulation development of high concentration monoclonal antibody dosage forms is the formation of proteinaceous subvisible and visible particulates during manufacturing and long-term storage. The level of proteinaceous and non-proteinaceous particulates in IgG formulations is an increasingly important part of purification and formulation development. (Refer Klaus Wuchner et al Journal of Pharmaceutical Sciences, vol. 99, no. 8, august 2010). Further the liquid formulation should be stable across different temperatures viz temperatures 2-8° C., 25° C., 40° C., and 55° C.

Many antibody preparations intended for human use require stabilizers to prevent denaturation, aggregation and other alternations to the proteins prior to the use of the preparation. Previously reported antibody Liquid antibody formulations (Lucentis, Avastin) had mannitol, trehalose as stabilizers. (Refer Susumu Uchiyama et al Biochimica Biophysica Acta 1844 (2014) 2041-2052; US20160137727; WO2009120684; U.S. Pat. No. 8,568,720). However trehalose is costly and not feasible from large scale process economics.

Also, the IV administration of antibody is usually given as an infusion rather than a bolus, and thus requires dilution of mAb formulation, including excipients into appropriate fluids suitable for IV administration. The resulting dilution of excipients, especially surfactants, which may decrease below the concentration required for prevention of aggregation during agitation, thereby resulting in generation of aggregates and subvisible particles following gentle agitation after dilution into PVC and PO IV bags containing 0.9% saline.

Hydrophobic interaction chromatography, ceramic hydroxyapatite and cation exchange resins have all been used for aggregate removal but none are ideal. Majority of previously reported antibody purification processes have heavily relied upon use of Hydrophobic interaction chromatography in combination with Protein A chromatography, Anion exchange chromatography, Cation exchange chromatography as a three or four step process (Refer WO2010141039, WO 2014/207763, WO2013066707, WO2015099165, WO2014102814, WO2015038888, WO2004087761). However, Hydrophobic interaction chromatography resins require large amounts of salts that are expensive, show low binding capacity, can be difficult to dispose of, and may not be compatible with the materials of construction of buffer and product holding tanks. Furthermore, the density difference between the buffers used for a HIC step can cause bed stability problems. Ceramic hydroxyapatite can also be used for the separation of aggregate from monomer, but the ceramic resin can be very difficult to unpack without damaging the resin. Therefore, storing the resin outside the column for re-use in a subsequent manufacturing campaign may not be possible (Refer Suzanne Aldington Journal of Chromatography B, 848 (2007) 64-78).

Three-step combinations of cation-exchange, anion-exchange flow through, hydrophobic interaction chromatography and mixed mode cation-exchange chromatography were found to deliver adequate clearance of host cell protein contaminants for a CHO derived monoclonal antibody. However, such purification schemes by-and-large have not caught on in commercial downstream operations due to the need to design the purification sequence separately for each mAb.

Thus, there is an urgent unmet need for an efficient platform process for antibody manufacturing and formulation that meets multiple criterion including robustness, reliability and scalability, in particular a platform that provides i) antibody titer of atleast 2 gm/L; ii) minimum aggregation/particulate formation across cell culture, purification and formulation processes; iii) improved purification showing optimal percentage recovery, high monomer content and minimum impurity levels; and iv) high concentration antibody formulation showing low viscosity, devoid of aggregation and sub-visible particles; thereby showing long-term stability.

### SUMMARY OF THE INVENTION

Applicant has surprisingly found
a) A feed composition and feeding strategy that takes into consideration nutrient consumption, by-product accumulation and the balance between promoting growth versus volumetric productivity wherein in particular, mammalian cell culture process parameters like use of particular basal medium, use of concentrated basal medium as feed solution, use of different feed solutions along-with a definite feeding strategy, maintaining lower concentrations of lactate and ammonia, are found to enhance cell growth, cell longevity and protein expression; thereby resulting in an increased antibody titer.
b) Specific salt concentration as part of buffer during Protein A affinity and Cation exchange steps that minimizes aggregation; thereby achieving a monomer content of greater than 99% with a recovery of greater than 80%.
c) Particle free liquid antibody formulations comprising of sucrose in combination with Histidine, Arginine, Polysorbate-80, Sodium chloride that impart higher potency and stability, reduces viscosity of highly concentrated antibody solutions at 2-8° C. for at least 9 months, at 25° C. for atleast 1 month, at 40° C. for atleast 42 days, at 55° C. for atleast 2 days as compared to formulation devoid of sucrose.

### LIST OF FIGURES

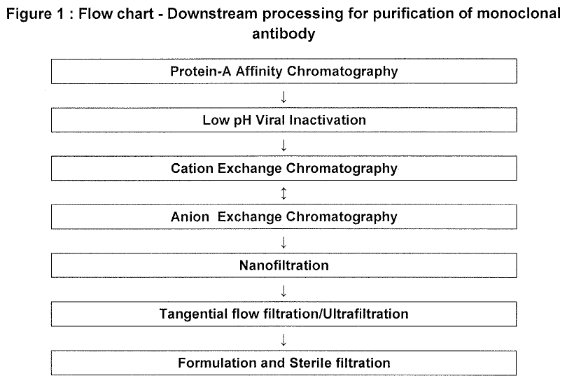

1. FIG. 1: Flow chart—Downstream processing for purification of monoclonal antibody

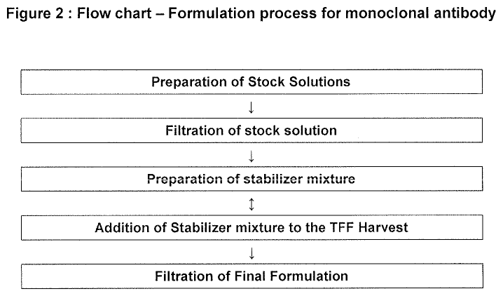

2. FIG. 2: Flow chart—Formulation process for monoclonal antibody

### DESCRIPTION OF THE INVENTION

Therapeutic proteins of the present invention include, but are not limited to antigen binding protein, humanized antibody, chimeric antibody, human antibody, bi-specific antibody, multivalent antibody, multi-specific antibody, antigen binding protein fragments, polyclonal, monoclonal, diabodies, nanobodies, monovalent, hetero-conjugate, multi-specific, auto-antibodies, single chain antibodies, Fab fragments, F(ab)′2, fragments, fragments produced by a Fab expression library, anti-idiotypic (anti-Id) antibodies, epitope-binding fragments and CDR-containing fragments or combination thereof.

In an embodiment of the present invention, the therapeutic protein is an antigen binding protein or immunoglobulin; more preferably is an IgG and most preferably is an IgG1 molecule. In first aspect of the present embodiment, immunoglobulin/antibody is a human IgG1 (G1m3 allotype) with a human kappa light chain specific to the Dengue virus epitope in domain III of the E protein. In second aspect of the present embodiment, the antibody is a fully human IgG1 monoclonal antibody specific to the rabies virus surface G glycoprotein. In third aspect of the present embodiment, the therapeutic protein can be selected from the group comprising of CTP19, CR57, CR4098, RVFab8, MabJA, MabJB-1, Mab 57, 17C7, 2B10, Ab513N/VIS513, N297Q-B3B9, Mab2E8, 2D22, DMScHuMab, 3CH5L1, HMB DV5, HMB DV6, HMB DV8, DB32-6, D88, F38, A48, C88, F108, B48, A68, A100, C58, C78, C68, D98, D188, C128, C98, A11, B11, R17D6, R14B3, R16C9, R14D6, R18G9, R16F7, R17G9, R16E5, antibodies derived from modification of 4E11A, adatacept, abciximab, adalimumab, aflibercept, alefacept, alemtuzumab, trastuzumab, basiliximab, bevacizumab, belatacept, bectumomab, certolizumab, cetuximab, daclizumab, eculizumab, efalizumab, entanercept, gemtuzumab, ibritumomab, infliximab, muromonab-CD3, omalizumab, palivizumab; panitumumab, pertuzumab, ranibizumab, rilonacept, rituximab, tositumomab, trastuzumab, zanolimab, nivolumab, pembrolizumab, hA20, AME-133, IMC-3G3, zalutumumab, nimmotuzumab, matuzumab, ch*)\hat{ }, KSB-102, MR1-1, SC100, SC101, SC103, muromonab-CD3, OKT4A, ibritumomab, gemtuzumab, motavizumab, infliximab, pegfilgrastin, CDP-571, etanercept, ABX-CBL, ABX-IL8, ABX-MAI pemtumomab, Therex, AS1405, natalizumab, HuBC-I, IDEC-131, VLA-I; CAT-152; J695, CAT-192, CAT-213, BR3-Fc, LymphoStat-B, TRAIL-RImAb, bevacizumab, omalizumab, efalizumab, MLN-02, HuMax-IL 15, HuMax-Inflam, HuMax-Cancer, HuMax-Lymphoma, HuMax-TAC, clenoliximab, lumiliximab, BEC2, IMC-ICI 1, DCIOI, labetuzumab, arcitumomab, epratuzumab, tacatuzumab, Cetuximab, MyelomaCide, LkoCide, ProstaCide, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-I, golimumab, CNTO 1275, CNTO 95, CNTO 328, mepolizumab, MORIOI, MORI 02, MOR201, visilizumab, HuZAF, volocixmab, ING-I, MLN2201, daclizumab, HCD 122, CDP860, PRO542, C 14, oregovomab, edrecolomab, etaracizumab, atezolizumab, iplimumab, mogamulizumab, lintuzumab, HuIDIO, Lym-1, efalizumab, ICM3, galiximab, eculizumab, obinutuzumab, pexelizumab, LDP-OI, huA33, WX-G250, sibrotuzumab, ofatumumab, Chimeric KW-2871, hu3S193, huLK26; bivatuzumab, raxibacumab, chl4.18, 3F8, BC8, huHMFGI, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-I, Pentacea, abagovomab, tositumomab, ustekinumab, 105AD7, GMAI 61, GMA321.

In other aspect of this embodiment, therapeutic protein is an antibody having binding affinity towards epitopes present on Dengue virus, Rabies virus, RSV, MPV, Influenza virus, Zika virs, West Nile virus, Yellow fever virus, chikungunya virus, HSV, CMV, MERS, Ebola virus, Epstein-Barr virus, Varicella-Zoaster virus, mumps virus, measles virus, polio virus, rhino virus, adenovirus, hepatitis A virus, Hepatitis B virus, hepatitis C virus, Norwalk virus, Togavirus, alpha virus, rubella virus, HIV virus, Marburg virus, Ebola virus, Human pappiloma virus, polyoma virus, metapneumovirus, coronavirus, VSV and VEE.

In another aspect of this embodiment, isoelectric point (pI) of said antigen binding protein is 7.5-8.5, more preferably about 7.8 to about 8.2, most preferably 8.12.

In particular, the antigen binding protein is a therapeutic, prophylactic or diagnostic antibody as described in WO2014025546, WO2015122995, WO2015123362, WO2006084006, WO2017027805 and WO2017165736, the contents of which are incorporated herein by reference in its entirety. More preferably, therapeutic protein is an antibody having 80% similarity to that VIS513 (Seq ID 1 or Seq ID 2). In other preferred aspect of the present embodiment, therapeutic protein is an antibody having more than 80% similarity to that of rabies monoclonal antibody (Seq ID 3 and Seq ID 4).

It is very well understood that any host may be used for the expression of therapeutic protein in the methods described herein. The cells may be wild or genetically engineered to contain a recombinant nucleic acid sequence, e.g. a gene, which encodes a polypeptide of interest (e.g., an antibody).

In second embodiment of the present invention, cell line used for the expression of therapeutic proteins is selected from the group including but not limited to CHO, CHOK1SV GS-KO, GS-CHO, CHO DUX-B11, CHO-K1, BSC-1, NSO myeloma cells, CV-1 in Origin carrying SV40 (COS) cells, COS-1, COS-7, P3X3Ag8.653, C127, 293 EBNA, MSR 293, Colo25, U937, SP2 cells, L cell, human embryonic kidney (HEK 293) cells, baby hamster kidney (BHK 21) cells, African green monkey kidney VERO-76 cells, HELA cells, VERO, BHK, MDCK, W138 cells, NIH-3T3, W138, BT483, Hs578T, HTB2, BT20, T47D, NS0 (a murine myeloma cell line that does not endogenously produce any immunoglobulin chains), CRL7O3O, HsS78Bst cells, PER.C6, SP2/0-Ag14, a myeloma cell line, a hybridoma cell line, human lung cells (W138), Retinal cells, human hepatoma line (Hep G2), and hybridoma cells.

In other aspect of the second embodiment, animal or mammalian host cells includes but not limited to Chinese hamster ovary cells (CHO) such as CHO-K1 (ATCC CCL-61), DG44 (Chasin et al., 1986, Som. Cell Molec. Genet., 12:555-556; and Kolkekar et al., 1997, Biochem., 36:10901-10909), SH87 cellICHO-DXB11 (G. Urlaub and L. A. Chasin, 1980 Proc. Natl. Acad. Sci., 77: 4216-4220. L. H. Graf, and L. A. Chasin 1982, Molec. Cell. Biol., 2: 93-96), CHO-K1 Tet-On cell line (Clontech), CHO designated ECACC 85050302 (CAMR, Salisbury, Wiltshire, UK), CHO clone 13 (GEIMG, Genova, IT), CHO clone B (GEIMG, Genova, IT), CHO-K1/SF designated ECACC 93061607 (CAMR, Salisbury, Wiltshire, UK), RR-CHOK1 designated ECACC 92052129 (CAMR, Salisbury, Wiltshire, UK), CHOK1sv (Edmonds et al., Mol. Biotech. 34:179-190 (2006)), CHO-S (Pichler et al., Biotechnol. Bioeng. 108:386-94 (2011)), dihydrofolate reductase negative CHO cells (CHO/−DHFR, Urlaub and Chasin, 1980, Proc. Natl. Acad. Sci. USA, 77:4216), and dp12.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293 cells subcloned for growth in suspension culture, Graham et al., 1977, J. Gen. Virol., 36:59); baby hamster kidney cells (BHK, ATCC CCL-10); CAP cell, AGE1.HN cell, monkey kidney cells (CV 1, ATCC CCL-70); African green monkey kidney cells (VERO-76, ATCC CRL-1587; VERO, ATCC CCL-81); mouse sertoli cells (TM4, Mather, 1980, Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TR1 cells (Mather, 1982, Ann. NY Acad. Sci., 383:44-68); MCR 5 cells; and FS4 cells.

In first aspect of the second embodiment, cell line used for the expression of therapeutic proteins is Chinese Hamster Ovary cells; more particularly the cell line is CHOK1SV GS-KO or GS-CHO.

In third embodiment of the present invention, the cells are cultivated in a batch, fed batch or continuous mode; more particularly in a fed batch mode. It is very well understood, that a person skilled in the art can modulate a process described in this invention according to available facilities and individual needs. More particularly, the cell culture process is carried out in fed batch mode providing enhanced cell growth, cell longevity and increased protein expression i.e. provides a harvest yield of atleast 2 gm/L, preferably in the range of 3 gm/L to about 6 gm/L.

In first aspect of the third embodiment, cell culture is conducted in a flask, a bioreactor, a tank bioreactor, a bag bioreactor or a disposable bioreactor. Preferably said bioreactor is selected from the group of stirred tank bioreactor, a bubble column bioreactor, an air lift bioreactor, a fluidized bed bioreactor or a packed bed bioreactor; and the said bioreactor has a volume selected from 1 L, 2 L, 3 L, 5 L, 10 L, 20 L, 100 L, 200 L, 250 L, 350 L, 500 L, 1000 L, 1500 L, 3000 L, 5000 L, 10000 L, 20000 L and 30,000 liters.

In second aspect of the third embodiment, the present cell culture media and methods may be used to increase antibody yield by about 5%, 10%, 15%, 20%, 30%, 40%, 50%, 60%, 70%, 80%, 90%, 100%, 110%, 120%, 130%, 140%, 150%, 160%, 180%, or 200%, most preferably about 40% to 60% as measured over a course of a fortnight. The time period of the fed batch method can be about 12 to 20 days; about 15 to 20 days or about 15 to 18 days.

In fourth embodiment of the present invention, cell culture medium is selected from the group comprising one or more of CD CHO, CD OptiCHO™, CD FortiCHO™ (Life Technologies); Ex-Cell™ CD CHO (Sigma Aldrich); ProCHOT™ 5 (Lonza); BalanCD™ CHO Growth A (Irvine Scientific); CDM4Mab (Hyclone); Celivento™ CHO-100 (EMD Millipore); Cell vento 200 (Merck Millipore); Cell vento 220 (Merck Millipore); Actipro (Hyclone); and combination thereof. Preferably the cell culture medium is selected from Cell Vento 220 (Merck), ACTIPRO (HyClone/GE), or Gibco™ Dynamis™ Medium (Thermo Fisher).

The cell culture medium is further supplemented with glucose and other feed solutions so as to increase cell growth, cell longevity, protein expression and yield. It is very well understood in the art that the feed solutions may be supplemented in rapid bolus or gradual drip manner.

The supplementation of feed solution in cell culture medium with a feeding strategy comprising of:
Initial feeding with Feed solution A, at 0.05% to 0.5% reactor volume, preferably at 0.1% to 0.2% reactor volume, from day 4;
Feeding with Feed solution A, at 0.1% to 0.5% reactor volume on day 6, 8, 10, 11 and 13;
Feed solution B at less than 8% reactor volume, from day 2 to day 14, on alternate day or in continuous manner;
Feeding with Feed solution C at less than 8% reactor volume for atleast 2 consecutive days beginning from day 2 or day 3, having an intermittent gap of 2 consecutive days, till day 12^{th} or day 14^{th} or day 15^{th} or day 16^{th} or day 18^{th}.
Feeding with Feed solution D at less than 0.5% reactor volume for atleast 2 consecutive days beginning from day 4 or day 5, on alternate day or in continuous manner or having an intermittent gap of 2 consecutive days, till day 12^{th} or day 14^{th} or day 15^{th} or day 16^{th} or day 18^{th}. Optionally feeding atleast one feed solution selected from EfficientFeed™ A, EfficientFeed™ B, EfficientFeed™ C, and Dow corning Antifoam C.
In a preferred aspect of fourth embodiment, said Feed solution A, Feed solution B, Feed solution C, Feed solution D is selected one or more from group comprising of Glucose, Cell Boost™ 5 Supplement (Hyclone), EX-CELL 293 (Sigma Aldrich), Cell Boost 7a and 7b supplements (Hyclone), 3× Actipro (Hyclone/GE), Cell Vento 220 (1× medium), EX-CELL® Advanced™ CHO Feed 1, EfficientFeed™ A, EfficientFeed™ B, and EfficientFeed™ C, and combination thereof.
In a most preferred aspect of the fourth embodiment, said Feed solution A is Cell Boost™ 5 Supplement (Hyclone); Feed solution B is EX-CELL 293 (Sigma Aldrich), Feed solution C is Cell Boost 7a supplements (Hyclone), Feed solution D is Cell Boost 7b supplements (Hyclone). Further, the cell culture medium is supplemented with 10% “3× Actipro” (Hyclone) on 3^{rd} day and 8% Cell Vento 220 (1× medium) on 7^{th} day of cell culturing. It is very well understood that all the feedings addition can vary by ±1% and ±1 day by a person skilled in the art.
Another aspect of the fourth embodiment includes cell culture conditions employed for increasing the cell growth and longevity and protein expression. The following cell culture conditions employed during the process includes but not limited to:
pH of cell culture medium is in the range of 6.5 and 7.5;
Osmolality of the culture medium is in the range of 250-500 mOsm/kg; more preferably 400-500 mOsm/kg.
Dissolved oxygen is in the range of 10-60%; preferably 20-40%; more preferably 30%.
Cell culture temperature is in the range of 30° C. to 38° C.; first temperature preferably 36-37° C. and optionally second temperature preferably 30-35° C.
Glucose concentration is maintained below 7%; preferably between 4% and 5%.
Harvesting the cell culture when viability is decreased to 80%;
Wherein the cell culture conditions are maintained in a manner that secondary metabolites such as Lactate concentration is not more than 5 g/L; and Ammonia concentration is not more than 5 mMol/L.
In fifth embodiment of the present invention, the said therapeutic protein obtained from the cell culture harvest is subjected to a purification process comprising of following steps i) affinity chromatography, ii) Viral inactivation, iii) ion exchange chromatography, and iv) filtration; wherein the overall process recovery is more than 70% and the final purified therapeutic protein has a purity/monomer content of atleast 90%, preferably more than 98%. Other impurities including residual cell DNA, residual cell protein, and residual protein A in final purified therapeutic protein is less than 1%.
In general aspect of the fifth embodiment, the inventors of this invention have succeeded in dealing with problem of aggregation of therapeutic protein during downstream processing of the said protein, by using i) Salt in a affinity chromatography wash step and ii) Linear gradient of salt solution for elution in ion exchange chromatography step. In preferred aspect of the said embodiment, the salt concentration of the buffers used in purification is in the range of 30 mM-500 mM, more preferably the salt concentration of the buffers used in purification is in the range of 50 mM-300 mM.
In first aspect of the fifth embodiment, affinity chromatography selected from the group comprising one or more of Protein A chromatography, Protein G chromatography, Protein L chromatography, and combination thereof; preferably the affinity chromatography used is Protein A chromatography.
In second aspect of the fifth embodiment, resin used for Protein A chromatography is selected from the group comprising one or more of Eshmuno A, KanCapA™, MabSelect SuRe™, MabSelect SuRe LX, MabSelect Xtra, rProtein A Sepharose Fast Flow, Poros® MabCapture A, Amsphere™ Protein A JWT203, ProSep HC, ProSep Ultra, and ProSep Ultra Plus; Preferrably the Protein A affinity chromatography resin is MabSelect SuRe™, Eshmuno A, Kancap A or Poros MabCapture; more preferably the Protein A affinity chromatography resin is MabSelect SuRe™.
In third aspect of the fifth embodiment, the wash buffer used for Protein A chromatography is selected from the group comprising one or more of
10-30 mM Phosphate buffer, preferably 20 mM Phosphate buffer; 100-150 mM NaCl, preferably 150 mM NaCl; 0.05% Polysorbate 80; pH 7.0±0.2.
10-30 mM Phosphate buffer, preferably 20 mM Phosphate buffer; 250 mM-1 M NaCl, preferably 1M NaCl; 0.05% Polysorbate 80; pH 7.0±0.2.
1-30 mM Phosphate buffer, preferably 10 mM Phosphate buffer; 100-150 mM NaCl, preferably 125 mM NaCl; 0.05% Polysorbate 80; pH 7.0±0.2.

In fourth aspect of the fifth embodiment, elution buffer used for Protein A chromatography comprises of 10-30 mM Citrate buffer; pH 3.0±0.5; and optionally 0.01-0.05% (w/v) Polysorbate 80; preferably the elution buffer comprises of 20 mM Citrate buffer; pH 3.0±0.2; and optionally 0.025% (w/v) Polysorbate 80.

In fifth aspect of the fifth embodiment, eluate obtained from the affinity chromatography step is subjected to viral inactivation and reduction. It is very well understood in the art that viral inactivation and reduction of the eluate may be effected by method selected individually or in combination from the group comprising of pH treatment, detergent treatment, heat treatment, and virus reduction filtration. In preferred aspect of this embodiment, the viral inactivation is effected by subjecting the eluate to low pH i.e. 3.3-3.5 for 50-100 minutes. Further, the eluate was pH neutralized by subjecting it to neutralization buffer i.e. 1 M Tris/Citrate buffer pH 7.0±0.2. It is very well understood in the art that any other compatible buffer may be used alternatively for effective pH neutralization of the eluate.

In sixth aspect of the fifth embodiment, the viral inactivated eluate is subjected to ion exchange chromatography. According to one of the aspect of this embodiment, ion exchange chromatography is cation exchange chromatography or anion exchange chromatography or their combination; and chromatography may be carried out in “bind and elute” mode or “flow through” mode. In preferred aspect of this embodiment, cation exchange chromatography and anion exchange chromatography is carried out in any sequential order. A further aspect of the fifth embodiment is that the said chromatography resin optionally is a multi-modal resin like Capto MMC resin (GE Healthcare).

In seventh aspect of the fifth embodiment, the viral inactivated eluate is subjected to cation exchange chromatography. In preferred aspect of this embodiment, the chromatography parameters including chromatography resin and buffer conditions are selected in such a manner that the positively charged therapeutic protein binds to the chromatography resin while the negatively charged molecules comes in the flow through, further therapeutic proteins are subjected to elution using a salt gradient. In preferred aspect of this embodiment, the cation exchange chromatography resin is selected from the group comprising one or more of sulfonate based group (e.g., MonoS, MiniS, Source 15S and 30S, SP SEPHAROSE® Fast Flow, SP SEPHAROSE® High Performance from GE Healthcare, TOYOPEARL® SP-650S and SP-650M from Tosoh, MACRO-PREP® High S from BioRad, Ceramic HyperD S, TRISACRYL® M and LS SP and Spherodex LS SP from Pall Technologies); a sulfoethyl based group (e.g., FRACTOGEL® SE, from EMD, POROS® S-10 and S-20 from Applied Biosystems); a sulphopropyl based group (e.g., TSK Gel SP 5PW and SP-5PW-HR from Tosoh, POROS® HS-20, HS 50, and POROS® XS from Life Technologies); a sulfoisobutyl based group (e.g., FRACTOGEL® EMD S03 “from EMD); a sulfoxy ethyl based group (e.g., SE52, SE53 and Express-Ion S from Whatman), a carboxymethyl based group (e.g., CM SEPHAROSE® Fast Flow from GE Healthcare, Hydrocell CM from Biochrom Labs Inc., MACRO-PREP® CM from BioRad, Ceramic HyperD CM, TRISACRYL® M CM, TRISACRYL® LS CM, from Pall Technologies, Matrex CELLUFINE® C500 and C200 from Millipore, CM52, CM32, CM23 and Express-Ion C from Whatman, TOYOPEARL® CM-650S, CM-650M and CM-650C from Tosoh); sulfonic and carboxylic acid based groups (e.g., BAKERBOND® Carboxy-Sulfon from J.T. Baker); a carboxylic acid based group (e.g., WP CBX from J.T Baker, DOWEX® MAC-3 from Dow Liquid Separations, AMBERLITE® Weak Cation Exchangers, DOWEX® Weak Cation Exchanger, and DIAION® Weak Cation Exchangers from Sigma-Aldrich and FRACTOGEL® EMD COO-from EMD); a sulfonic acid based group (e.g., Hydrocell SP from Biochrom Labs Inc., DOWEX® Fine Mesh Strong Acid Cation Resin from Dow Liquid Separations, UNOsphere S, WP Sulfonic from J.T. Baker, SARTOBIND® S membrane from Sartorius, AMBERLITE® Strong Cation Exchangers, DOWEX® Strong Cation and DIAION® Strong Cation Exchanger from Sigma-Aldrich); and a orthophosphate based group (e.g., PI 1 from Whatman). In most preferred aspect of this embodiment, the resin used for cation exchange chromatography is Fractogel® EMD SO₃⁻, Fractogel® EMD SE Hicap (Merck), CMM HyperCel™ (Pall Corporation), Capto S ImpAct. In another aspect of fifth embodiment, process parameters for cation exchange chromatography includes but not limited to Pre-equilibration buffer [200 mM Citrate buffer; pH 6.0±0.2]; Equilibration buffer [10 mM Citrate buffer; Polysorbate 80 (0.025% (w/v)); pH 6.0±0.2]; Low pH hold for neutralization; Wash Buffer A [10 mM Citrate buffer; pH 6.0±0.2]; Wash buffer B [20 mM Citrate buffer; 300-500 mM NaCl; pH 6.0±0.2]; CIP buffer [0.5M NaOH]; Residence time [4.00-7.00 minutes]; Column used [XK26].

In eighth aspect of the fifth embodiment, the viral inactivated eluate is subjected to anion exchange chromatography. In preferred aspect of this embodiment, the chromatography parameters including chromatography resin and buffer conditions are selected in such a manner that all negatively charged impurities are bound with the membrane while the therapeutic protein elutes in a flow through. In preferred aspect of this embodiment, the anion exchange chromatography resin is selected from the group comprising one or more of DEAE cellulose, POROSO PI 20, PI 50, HQ 10, HQ 20, HQ 50, D 50 from Applied Biosystems, SARTOBIND® Q from Sartorius, MonoQ, MiniQ, Source 15Q and 30Q, Q, DEAE and ANX SEPHAROSE® Fast Flow, Q SEPHAROSE, Q SEPHAROSE® High Performance, QAE SEPHADEX® and FAST Q SEPHAROSE® (GE Healthcare), WP PEI, WP DEAM, WP QUAT from J.T. Baker, Hydrocell DEAE and Hydrocell QA from Biochrom Labs Inc., U Osphere Q, MACRO-PREP® DEAE and MACRO-PREP® High Q from Biorad, Ceramic HyperD Q, ceramic HyperD DEAE, TRISACRYL® M and LS DEAE, Spherodex LS DEAE, QMA SPHEROSIL® LS, QMA SPHEROSIL® M and MUSTANG® Q from Pall Technologies, DOWEX® Fine Mesh Strong Base Type I and Type II Anion Resins and DOWEX® MONOSPHER E 77, weak base anion from Dow Liquid Separations, INTERCEPT® Q membrane, Matrex CELLUFINE® A200, A500, Q500, and Q800, from Millipore, FRACTOGEL® EMD TMAE, FRACTOGEL® EMD DEAE and FRACTOGEL® EMD DMAE from EMD, AMBERLITE® weak strong anion exchangers type I and II, DOWEX® weak and strong anion exchangers type I and II, DIAION® weak and strong anion exchangers type I and II, DUOLITE® from Sigma-Aldrich, TSK gel Q and DEAE 5PW and 5PW-HR, TOYOPEARL® SuperQ-650S, 650M and 650C, QAE-550C and 650S, DEAE-650M and 650C from Tosoh, QA52, DE23, DE32, DE51, DE52, DE53, Express-Ion D and Express-Ion Q from Whatman; more preferably Anion-exchange chromatography resin is selected from Sartobind Q (Sartorius), Eshmuno Q (Merck), MUSTANG® Q (Pall Corporation) and Poros X (Thermo). In another aspect of fifth embodiment, process parameters for anion exchange chromatography includes but not limited to Cleaning buffer [0.5M NaOH]; Pre-equilibration buffer [200 mM Citrate buffer; pH 6.0±0.2]; Equilibration buffer [20 mM Citrate buffer; pH 6.0±0.2; and optionally 0.025% Polysorbate 80]; Storage buffer [0.1M NaOH]; Linear Flow rate [10-500 cm/hr, more particularly 100-150 cm/hr]; Column used [XK26].

The purification process of aforementioned embodiments can further comprise of atleast one additional chromatography step selected from the group comprising one or more of Hydrophobic interaction chromatography, Hydrophobic charge induction chromatography, Ceramic hydroxyapatite chromatography, Multimodal chromatography (Capto MMC and Capto Adhere), Membrane chromatography (Q membranes including Intercept™ (Millipore), Mustang® (Pall Corporation) and Sartobind™ (Sartorius)).

In ninth aspect of the fifth embodiment, virus particles were removed by using 20 nm filter. The filter used for removal of viral particles includes but not limited to virus retentive filter selected from the group of Viresolve PRO (Merck), Planova 20N (Asahi Kasei), Bio EXL PALL PEGASUS PRIME, PEGASUS SV4 (Pall Life Sciences), and Virosart (Sartorius), Virosart CPV filter from Sartorius, Virosolve from Millipore, Ultipor DV20 or DV50 from Pall, Planova 20N and 50N or BioEx from Asahi. It is very well understood in the art that any other filter having retention capacity for viruses may be used in this step; preferably the filter used for removal of viral particles is selected from Viresolve PRO (Merck), Bio EXL PALL PEGASUS PRIME, PEGASUS SV4 (Pall Life Sciences), and Virosart (Sartorius).

In tenth aspect of the fifth embodiment, the therapeutic protein is concentrated to a desired concentration and buffer exchanged in formulation buffer. The buffer is exchanged in a tangential flow filtration system or an ultra flow filtration system. The other parameters of Tangential flow filtration comprises of one or more selected from Diafilteration using diafilteration buffer [25 mM Histidine buffer; 75 mM Arginine buffer; 50-150 mM NaCl; pH 6.50±0.5]; Cleaning buffer [0.5M NaOH]; Storage buffer [0.1M NaOH]; Equilibration using 5-10× membrane volume; Concentration and Diafilteration using 10-20 diafilteration volume; WFI wash using 3-5 membrane volume; cleaning using 0.5-1.0 M NaOH; Storage [0.1M NaOH]. In one of the preferred aspect of this embodiment, Tangential flow filtration is carried out using 30 kDa MWCO membrane selected from the group comprising one or more of Centramate T series PES membrane (Pall Corporation), Hydrosart (Sartorius), and Pelicon 3 (Merck).

In sixth embodiment of the present invention, the said purified therapeutic protein is formulated with pharmaceutical excipients, wherein the osmolality of the formulation is in the range of 300 mOsm/Kg to 500 mOsm/Kg and viscosity of the formulation is less than 2.5 mPa-S.

In first aspect of the sixth embodiment, therapeutic protein formulation comprises of atleast one antigen binding protein, atleast one stabilizer, atleast one buffering agent, atleast one tonicity agent, and atleast one surfactant. Optionally, formulation comprises of a preservative.

In second aspect of the sixth embodiment, stabilizer is an carbohydrate. Stabilizer is selected from the group comprising of one or more of sucrose, sorbitol, trehalose, mannitol, dextran, inositol, glucose, fructose, lactose, xylose, mannose, maltose, Raffinose and combination thereof; more preferably the stabilizer is sucrose. In yet another aspect of this embodiment, stabilizer comprises of sucrose at a concentration of about 0.1% to about 2.5% w/v, preferably <1% sucrose w/v.

In third aspect of the sixth embodiment, buffering agent is selected from the group comprising of one or more of histidine, arginine, glycine, sodium citrate, sodium phosphate, citric acid, HEPES, potassium acetate, potassium citrate, potassium phosphate, sodium acetate, sodium bicarbonate, Tris base, or Tris-HCl, and combination thereof. Preferably, buffering agent provides a pH of about 5.5 to 7.5, about 6.0 to 7.0, about 6.3 to about 6.8, or about 6.5

In fourth aspect of sixth embodiment, buffering agent is Histidine. In preferred aspect of this embodiment, the buffering agent comprises Histidine at a concentration of about 5 mM to about 150 mM, about 10 mM to about 50 mM, about 20 mM to about 40 mM. In most preferred aspect of this embodiment, buffering agent comprises Histidine at a concentration of about 25 mM.

In fifth aspect of sixth embodiment, buffering agent is Arginine. In preferred aspect of this embodiment, the buffering agent comprises Arginine at a concentration of about 5 mM to about 200 mM, about 50 mM to about 150 mM, about 50 mM to about 100 mM. In most preferred aspect of this embodiment, buffering agent comprises Arginine at a concentration of about 70 to 80 mM.

In sixth aspect of sixth embodiment, tonicity agent is selected from the group comprising of one or more of sodium chloride, dextrose, glycerin, mannitol, and potassium chloride. In preferred aspect of this embodiment, tonicity agent comprises of Sodium Chloride and is present at a concentration of about 10 mM to about 500 mM; preferably at concentration of about 50 mM to about 250 mM; most preferably at a concentration of about 100-145 mM.

In seventh aspect of sixth embodiment, surfactant is present at a concentration of about 0.001 to about 0.2% (w/v); and is selected from the group comprising of one or more of polysorbates (e.g. polysorbate-20 or polysorbate-80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl-or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT® series (Mona Industries, Inc., Paterson, N.J.), polyethyl glycol, polypropyl glycol, and copolymers of ethylene and propylene glycol (e.g. Pluronics, PF68 etc). In preferred aspect of this embodiment, surfactant comprises of Polysorbate 80 and is present at a concentration of about 0.001% to about 0.2% w/v; preferably at concentration of about 0.002% to about 0.02%; about 0.005% to about 0.02%, most preferably at a concentration of about 0.02%.

In eighth aspect of sixth embodiment, the formulation comprises of a therapeutic protein at a concentration of about 1 mg/L to about 150 mg/L, about 1 mg/L to about 50 mg/L, about 20 mg/L to about 40 mg/L. Preferably the formulation comprises of a therapeutic protein at a concentration of about 1 mg/L to about 50 mg/L.

In ninth aspect of the sixth embodiment, the formulation further comprises of preservative, the preservative may be selected from the group comprising of benzyl alcohol, m-cresol, and phenol.

In seventh embodiment of the present invention, the therapeutic protein formulation comprises of atleast one therapeutic protein, sucrose, arginine, histidine, Sodium chloride, Polysorbate 80. Preferably therapeutic protein formulation comprises of about 1 mg/ml to about 50 mg/ml of therapeutic protein; about 20 mM to about mM mg/ml of Histidine; about 50 mM to about 100 mM of Arginine; about 0.002% to about 0.02% Polysorbate 80 (w/v); about 50 mM to about 150 mM NaCl; and <2.5% Sucrose w/v. The pH of the formulation is in the range of 6.0 to about 7.0 and Osmolality of the formulation is in the range of 300 mOsm/Kg to about 450 mOsm/Kg.

In one of the preferred aspect of seventh embodiment, a pharmaceutical formulation comprises of 2-80 mg/ml of Dengue monoclonal antibody; 25 mM of Histidine; 75 mM of Arginine; 101 mM NaCl; 0.02% Polysorbate 80 (w/v); and 0.5% Sucrose w/v; wherein pH of the formulation is 6.5±0.5 Osmolality 380 mOsm/Kg, viscosity less than 2.5 mPa-S.

In one of the preferred aspect of seventh embodiment, a pharmaceutical formulation comprises of 25 mg/ml of Dengue monoclonal antibody; 25 mM of Histidine; 75 mM of Arginine; 101 mM NaCl; 0.02% Polysorbate 80 (w/v); and 0.5% Sucrose w/v; wherein pH of the formulation is 6.5±0.5, Osmolality 380 mOsm/Kg, viscosity less than 2.5 mPa-S.

In one of the preferred aspect of seventh embodiment, a pharmaceutical formulation comprises of 50 mg/ml of Dengue monoclonal antibody; 25 mM of Histidine; 75 mM of Arginine; 101 mM NaCl; 0.02% Polysorbate 80 (w/v); and 0.5% Sucrose; wherein pH of the formulation is 6.5±0.5 Osmolality 380 mOsm/Kg, viscosity less than 2.5 mPa-S.

In one of the preferred aspect of seventh embodiment, a pharmaceutical formulation comprises of 2-80 mg/ml of Rabies monoclonal antibody; 25 mM of Histidine; 75 mM of Arginine; 101 mM NaCl; 0.02% Polysorbate 80 (w/v); and 0.5% Sucrose w/v; wherein pH of the formulation is 6.5±0.5 Osmolality 380 mOsm/Kg, viscosity less than 2.5 mPa-S.

In one of the preferred aspect of seventh embodiment, a pharmaceutical formulation comprises of 25 mg/ml of Rabies monoclonal antibody; 25 mM of Histidine; 75 mM of Arginine; 101 mM NaCl; 0.02% Polysorbate 80 (w/v); and 0.5% Sucrose w/v; wherein pH of the formulation is 6.5±0.5 Osmolality 380 mOsm/Kg, viscosity less than 2.5 mPa-S.

A pharmaceutical formulation comprising of 50 mg/ml of Rabies monoclonal antibody; 25 mMof Histidine; 75 mMof Arginine; 101 mM NaCl; 0.02% Polysorbate 80 (w/v); and 0.5% Sucrose w/v; wherein pH of the formulation is 6.5±0.5 Osmolality 380 mOsm/Kg, viscosity less than 2.5 mPa-S.

According to another aspect of the seventh embodiment, said pharmaceutical formulation of antibody could be a lyophilized formulation.

In eighth embodiment of the present invention, the affinity and potency of the therapeutic protein is measured by one or more of ELISA or flow cytometry. In preferred aspect of the eighth embodiment, indirect ELISA based method is used to quantify binding of therapeutic protein to the specific antigen. In preferred aspect of this embodiment, Dengue Mab formulation is tested against all serotypes of the dengue viruses and amount of Dengue mAb is determined. The potency of the therapeutic protein is reported as % activity relative to the reference standard. It is very well understood that any other similar method may be used to demonstrate the potency and affinity of the therapeutic protein.

In ninth embodiment of the present invention, focus reduction neutralization test (PRNT/FRNT) or a related test is carried out for evaluating neutralization of viral activity by therapeutic protein. In preferred aspect of this embodiment, Dengue mAb formulation is tested against all serotypes of the dengue viruses and EC50 values are calculated for neutralization of Dengue Viruses. It is very well understood that any other similar method may be used to demonstrate the neutralization activity of the therapeutic protein.

In tenth embodiment of the present invention, HPLC based size exclusion chromatography is used to assess the presence of aggregates in therapeutic protein formulation. In preferred aspect of this embodiment, Phenomenex Bio-Sec-S 3000 column is used to demonstrate the aggregate and monomer percentage of Dengue mab formulation. It is very well understood that any other similar method may be used to assess the presence of aggregates in therapeutic protein formulation.

In eleventh embodiment of the present invention, the formulation may be stored in a suitable container. The container may be selected from a bottle, a vial, a IV bag, a wearable injector, a bolus injector, a syringe, a pen, a pump, a multidose needle syringe, a multidose pen, a injector, a syrette, an autoinjector, a pre-filled syringe, or a combination thereof.

At least one primary packaging component comprises a container closure selected from polypropylene (PP), polyethylene terephthalate (PETG), high-density polyethylene (HDPE), polyethylene terephthalate (PET), polypentafluorostyrene (PFS), polycarbonate, polyvinyl chloride (PVC), polyolefin, polycyclopentane (CZ®), cyclic olefin copolymer (COC), and combinations or copolymers thereof.

The anti-dengue antibody or anti-rabies antibody formulations disclosed herein can be used (alone or in combination with other agents or therapeutic modalities) to treat, prevent and or diagnose dengue or rabies virus. For example, the combination therapy can Include an anti-dengue antibody molecule co-formulated with, and/or co-administered with, one or more additional therapeutic agents, e.g., antiviral agents (Including other anti-dengue antibodies), vaccines (Including dengue virus vaccines), or agents that enhance an immune response. In other embodiments, the antibody molecules are administered in combination with other therapeutic treatment modalities, such as Intravenous hydration, fever-reducing agents (such as acetaminophen), or blood transfusion. Such combination therapies may advantageously utilize lower dosages of the administered therapeutic agents, thus avoiding possible toxicities or complications associated with the various monotheraples.

### EXAMPLES

### Example 1: Upstream Process for Cell Culturing and Expression of Therapeutic Protein i.e. Dengue (VIS513) Monoclonal Antibody

Protocol:

Cell culturing at 10 L scale was carried out in a Fed batch manner using below mentioned parameters during fermentation/upstream process.

The Dengue monoclonal antibody was expressed in cell line “CHO-K1 SV GS-KO” obtained from Visterra Inc. USA.
Cell culture medium used for cell growth and expression of therapeutic protein i.e. Dengue monoclonal antibody was 1× Celivento™ CHO-220 Liquid Medium.
Feed solution A, Feed solution B, Feed solution C, Feed solution D selected from group comprising of Glucose, Cell Boost™ 5 Supplement (Hyclone), EX-CELL 293 (Sigma Aldrich), Cell Boost 7a and 7b supplements (Hyclone), 3× Actipro (Hyclone), Cell Vento 220 (3× medium), EX-CELL® Advanced™ CHO Feed 1 was used for supplementation feed.
pH of the fermentation medium was maintained at 6.7 to 7.5.
Osmolality of the fermentation medium was maintained at <490 mOsm/Kg.
Dissolved oxygen of the fermentation medium was maintained at about 20% to about 40%.
Temperature of the fermentation medium was maintained at 36.5±0.5.
The culture was harvested upon drop in cell count to 60%

Feed supplementation was done in a gradual drip manner as per following Table 1:

TABLE 1

| Feeding | | | | | Basal | Basal |
|---|---|---|---|---|---|---|
| Strategy | | | | | Medium 1 | Medium 2 |
| protocol | Feed | Feed | Feed | Feed | (3X Actipro″ | (Cellvento ™ |
| Day | A | B | C | D | (Hyclone) | CHO-220 (3X)) |
| 0 | | | | | | |
| 1 | | | | | | |
| 2 | | 4% | | | | |
| 3 | | | | | 10% | |
| 4 | 0.2% | | 4% | 0.4% | | |
| 5 | | 6% | 4% | 0.4% | | |
| 6 | 0.2% | 4% | 4% | 0.4% | | |
| 7 | | 6% | | | | 8% |
| 8 | 0.2% | | 4% | 0.4% | | |
| 9 | | 4% | 4% | 4.0% | | |
| 10 | 0.1% | 2% | 4% | 0.4% | | |
| 11 | 0.2% | 2% | 2% | 0.2% | | |
| 12 | | 4% | | | | |
| 13 | 0.1% | 2% | 2% | 0.2% | | |
| 14 | | 3% | | | | |
| 15 | | 3% | | | | |
| 16 | | | | | | |

Results & Conclusion:

Viable colony count and Yield obtained during the fermentation process was as follows:

TABLE 2
Viable colony count and Yield during the fermentation process

| | Batch 1 | | | |
|---|---|---|---|---|
| | Viable | | Batch 2 | |
| Day | colony count | Titer gm/L | Viable colony count | Titer gm/L |
| 0 | 0.8 | | 0.81 | |
| 1 | 1.5 | | 1.7 | |
| 2 | 2.8 | | 3.45 | |
| 3 | 4.3 | | 5.9 | |
| 4 | 6.5 | | 7.4 | |
| 5 | 8.4 | | 11.8 | |
| 6 | 10.1 | 0.54 | 15.5 | 0.63 |
| 7 | 12.7 | 0.77 | 18.5 | 0.91 |
| 8 | 14.5 | 1.18 | 19 | 1.29 |
| 9 | 17 | 1.62 | 18.5 | 1.76 |
| 10 | 17.25 | 2.15 | 18.8 | 2.42 |
| 11 | 17.25 | 2.64 | 18 | 2.85 |
| 12 | 16.8 | 3.05 | 28 | 3.39 |
| 13 | 15.3 | 3.6 | 17.2 | 4.0 |
| 14 | 15 | 3.99 | 17 | 4.32 |
| 15 | 14.7 | 4.53 | 14.9 | 4.68 |
| 16 | 14.5 | 4.56 | 13.5 | 4.86 |

Applicant has found that by using Cell culture process comprising of basal medium, concentrated basal medium as feed solution, use of feed solutions along-with a definite feeding strategy, enhanced cell growth, lower concentrations of lactate and ammonia can be obtained thereby effectively maintaining the cell count and increasing cell longevity and high yield. Yield of greater than 4 gm/L was obtained in fermentation process. Harvest obtained was further subjected to purification/downstream processing.

### Example 2

Cell culture obtained in Example 1 was harvested and later subjected to protocol for purification of the dengue (VIS513) monoclonal antibody as per FIG. 1

Detailed process used was as follows:
Protein-A Affinity Chromatography:
   In this step targeted monoclonal antibody was separated from the media components in the harvested supernatant. Clarified supernatant was passed through the chromatography column and then eluted using compatible elution buffers.
   Materials used:
Resin (Matrix): Mab Select Sure/Eshmuno A (Protein-A Affinity)
Residence Time: 4.0-8.0 minutes
Column used: XK 26
Equilibration Buffer: 20 mM Phosphate Buffer+150 mM NaCl+0.05% (w/v) Polysorbate 80, pH 7.0±0.2.
Wash I Buffer: 20 mM Phosphate Buffer+150 mM NaCl+0.05% (w/v) Polysorbate 80, pH 7.0±0.2.
Wash II Buffer: 20 mM Phosphate Buffer+1M NaCl+0.05% (w/v) Polysorbate 80, pH 7.0±0.2.
Wash III Buffer: 10 mM Phosphate Buffer+125 mM NaCl+0.025% (w/v) Polysorbate 80, pH 6.0±0.2.
Elution Buffer: 20 mM Citrate buffer+0.025% (w/v) Polysorbate 80, pH 3.0±0.2.
CIP Buffer: 0.1 M NaOH

Process Parameters Used:

TABLE 3

| | | | Linear Flow Rate |
|---|---|---|---|
| S. No. | Process Step | Column Volume | (cm/hr) |
| 1 | Equilibriation | 5 | <300 |
| 2 | Loading (mL) | As Actual | <300 |
| 3 | Wash I | 2 | <300 |
| 4 | Wash II | 4 | <300 |
| 5 | Wash III | 4 | <300 |
| 6 | Elution | 5 | <300 |
| 7 | Cleaning | 3 | <300 |
| 8 | Storage | 2 | <300 |

1. Low pH Viral Inactivation
i. Eluate from protein-A Affinity chromatography was subjected to low pH i.e. 3.5±0.1 for 60±10 minutes to inactivate the viral particles.
ii. After low pH hold, eluate was neutralized using neutralization buffer i.e. 1 M Tris/Citrate buffer having pH 7.0±0.2.
iii. Conductivity of neutralized eluate was adjusted using WFI with 0.025% (w/v) Polysorbate 80.

2. Cation Exchange Chromatography

Positively charged antibody molecules bound with the column while negatively charged molecules come in the flow through. Column bound antibody molecules are eluted using salt gradient.

Materials Used:

Resin used: Fractogel SO3⁻/Fractogel SE Hicap (Merck)

Residence Time: 4.00-7.00 minutes

Column used: XK 26

Pre Equilibration: 200 mM Citrate buffer pH 6.0±0.2.

Equilibration: 10 mM Citrate buffer+0.025% (w/v) Polysorbate 80, pH 6.0±0.2.

Loading: Low pH Hold neutralized.

Wash Buffer A: 10 mM Citrate buffer, pH 6.0±0.2.

Wash Buffer B: 20 mM Citrate buffer+300 mM NaCl, pH 6.0±0.2.

CIP Buffer: 0.5 M NaOH

Storage Buffer: 0.1 M NaOH

Process Parameters:

TABLE 4

| | | | Linear Flow Rate |
|---|---|---|---|
| S. No. | Process Step | CV | (cm/hr) |
| 1 | Pre Equilibration | 2 | <300 |
| 2 | Equilibration | 5 | <300 |
| 3 | Loading | As Actual | <300 |
| 4 | Wash | 5 | <300 |
| 5 | Elution 0-60% (Gradient) | 15 | <300 |
| 6 | 100% B | 2 | <300 |
| 7 | GIP | 3 | <300 |

Fraction Collection During Gradient

TABLE 5

| S. No. | Fraction name | Collection Criteria (UV₂₈₀) mAU |
|---|---|---|
| 1 | Fraction 01 | Upto 300 |
| 2 | Fraction 02 | 300-700 |
| 3 | Fraction 03 | 700 till bas eline |
| 4 | Fraction 04 (100% B) | Baseline to base line |

Anion Exchange Chromatography:

All negatively charged impurities are bound with the membrane while antibody comes in the flow through.

Materials Used:

Membrane/Resin used: Sartobind Q single Sep mini (Sartorius)/Eshmuno Q

Loading volume: 150 mg/mL-1000 mg/mL

Column used: XK 26

Cleaning Buffer: 0.5 M NaOH

Pre-equilibration buffer: 200 mM Citrate buffer, pH 6.0±0.2.

Equilibration Buffer: 20 mM Citrate buffer pH 6.0±0.2; and optionally 0.025% PS-80 pH 6.0±0.2

Storage Buffer: 0.1 M NaOH

Process Parameters:

TABLE 6

| S. No. | Process Step | Column Volume (CV) | Linear Flow Rate (cm/hr) |
|---|---|---|---|
| 1 | Cleaning | 10 | <300 |
| 2 | Pre Equilibration | 10 | <300 |
| 3 | Equilibration | 20 | <300 |
| 4 | Loading | As Actual | <300 |
| 5 | Post Load Wash | 20 | <300 |
| 6 | Cleaning | 10 | <300 |

3. Nano-Filtration:

20 nm nanofilter i.e. Viresolve PRO (Merck), was used to remove any virus particles available in the therapeutic protein.

4. Tangential Flow Filtration/Ultra Flow Filtration:

The antibody was concentrated to desired concentration and buffer exchanged in one of the three formulation buffers.

Material Used:

Formulation Buffer:
Buffer 1: 25 mM Histidine buffer+75 mM Arginine Buffer+75 mM NaCl, pH 6.50+0.25;
Buffer 2: 25 mM Histidine, 75 mM Arginine, 101 mM NaCl;
Buffer 3: 25 mM Histidine, 75 mM Arginine, 75 mM to 101 mM NaCl, Polysorbate-80 0.002% w/v
Cleaning Buffer: 0.5 M NaOH

Storage Buffer: 0.1 M NaOH

Membrane used: PALL Centramate T Series, PES membrane MWCO: 30 kDa

Process Parameters:

TABLE 7

| S. | | | |
|---|---|---|---|
| No. | Process Step | Description | Remark |
| 1 | Cleaning | 0.5 M NaOH | 30 Minutes recirculation |
| 2 | WFI Cleaning | WFI, till conductivity | — |
| | | come below 1.3 | |
| | | μS/cm | |
| 3 | Equilibration | 400 ml | — |
| 4 | Concentration and | ~10-12 DV pass | — |
| | Diafiltration | | |
| 5 | WFI | WFI was 1000 mL | — |
| 6 | Cleaning | 0.5 M NaOH | 30 Minutes recirculation |

Sterile Filtration

Stabilizer was added to the antibody solution and sterile filtered through 0.2μ filter.

Results:

Stage wise recovery of the various steps used in the purification process.

TABLE 8

| S. No. | Stage | Recovery (%) | Purity (%) |
|---|---|---|---|
| 1 | Protein-A Affinity Chromatography | 98 | 99.3 |
| 2 | Low pH Viral Inactivation | 98 | — |
| 3 | Cation Exchange Chromatography | 90 | 99.52 |
| 4 | Anion Exchange Chromatography | 95 | 99.46 |
| 5 | Nanofiltration | 100 | — |
| 6 | TFF/UFF | 98 | — |
| 7 | Formulation and Sterile filtration | 100 | — |

The overall process recovery was found to be ˜80% and overall purity was found to be >99%.

TABLE 9
Impurity data

| Assay | Accepted Criterion | Batch 1 | Batch 2 |
|---|---|---|---|
| Purity by HP-SEC (% | Monomer should be | 99.74 | 99.11 |
| monomer | >90.00%. | | |
| | Retention time of monomer | | |
| | should be comparable to | | |
| | reference standard | | |
| Residual CHO DNA | <2 pg/mg IgG | 0.005 | 0.004 |
| (pg/mg of mAb) | | | |
| Residual Protein A | ≤10.00 ng/mg IgG | 1.05 | 0.82 |
| (ng/mg of mAb) | | | |
| Residual CHO Protein | ≤100.00 ng/mg IgG | 1.74 | 3.80 |
| (ng/mg of mAb) | | | |
| Endotoxin (EU/mg of | ≤0.1 EU/mg of protein | <0.05 | <0.05 |
| protein) | | | |

TABLE 10
Batch wise Recovery & Purity

| S. No. | Batch No. | Recovery (%) | Purity (%) |
|---|---|---|---|
| 1 | Batch 1 | 85 | 99.21 |
| 2 | Batch 2 | 87 | 99.26 |

### Example 3

Purified Dengue (VIS513) monoclonal antibody was formulated as follows:

Excipients i.e. Arginine, Histidine, NaCl, Sucrose, and polysorbate-80 were added and mixed thoroughly using a magnetic stirrer at 50-60 RPM to form a mixture of excipients. This mixture was then added into the Dengue mAb TFF harvest gradually with stirring rate 50-60 RPM. pH was checked (pH 6.5) and if required adjusted by histidine-arginine buffer. The final formulation was filtered through a 0.2 μM filter and filled into final container.

The concentration of each component in the final formulation was as follows:

TABLE 11

| Ingredient | Formulation 1 | | Formulation 2 | | Formulation 3 | |
|---|---|---|---|---|---|---|
| Dengue Mab (VIS513) | 10 | mg/ml | 25 | mg/ml | 50 | mg/ml |
| Histidine | 25 | mM | 25 | mM | 25 | mM |
| Arginine | 75 | mM | 75 | mM | 75 | mM |
| Sodium Chloride | 101 | mM | 101 | mM | 101 | mM |
| Sucrose | 0.5% | w/v | 0.5% | w/v | 0.5% | w/v |
| Polysorbate-80 | 0.02% | w/v | 0.02% | w/v | 0.02% | w/v |
| pH | 6.5 + 0.5 | | 6.5 + 0.5 | | 6.5 + 0.5 | |
| Osmolality | 380 | mOsm/kg | 380 | mOsm/kg | 380 | mOsm/kg |

These formulations were further tested for purity, stability, efficacy and potency for 9 months.

### Example 4

Effect of presence of Sucrose in VIS513 Dengue antibody formulation was studied for testing potency by ELISA assay on EDIII protein of DV1. The formulation studies were done for temp. 2-8° C., 25° C., and 40° C.

Results:

1. VIS513 Dengue Antibody Formulation without Sucrose

TABLE 12

| | % Potency compared to Reference | | | |
|---|---|---|---|---|
| | Standard Stored at 2-8° C. | | | |
| | 2-8° C. | | RT (25° C.) | 40° C. |
| Ingredient (QTY) | 15 Days | 30 Days | 15 Days | 30 Days |
| L- Histidine 25 mM | 78.8 | 73.2 | 79.8 | 53.8 |
| L- Arginine 75 mM | | | | |
| Sodium chloride 75 mM | | | | |
| Polysorbate 80 0.02% w/v | | | | |

2. VIS513 Dengue Antibody Formulation with Sucrose

TABLE 13

| | % Potency compared to Reference | | | |
|---|---|---|---|---|
| | Standard Stored at 2-8° C. | | | |
| | 2-8° C. | | RT (25° C.) | 40° C. |
| Ingredient (QTY) | 15 Days | 30 Days | 15 Days | 30 Days |
| L- Histidine 25 mM | 90.5 | 86.3 | 94.6 | 81.6 |
| L- Arginine 75 mM | | | | |
| Sodium chloride 101 mM | | | | |
| Polysorbate 80 0.02% w/v | | | | |
| Sucrose 0.5% w/v | | | | |

Reference Standard Formulation Composition:

| Ingredient | (QTY) | |
|---|---|---|
| L- Histidine | 25 | mM |
| L- Arginine | 75 | mM |
| Sodium chloride | 101 | mM |
| Polysorbate 80 | 0.02% | w/v |
| Sucrose | 0.5% | w/v |

Conclusion: Addition of 0.5% w/v improves stability as compared to the corresponding sampling point without sucrose.

### Example 5

VIS513 antibody formulation was stored at 40° C. for 20 days and later potency of VIS513 was evaluated by ELISA test. Effect of increasing Sucrose Strength was studied on VIS513 antibody formulation at 40° C., wherein sucrose concentration of 0.1, 0.2 and 0.5% was evaluated.

Results:

TABLE 14

| | % Potency compared to Reference |
|---|---|
| Ingredient (QTY) | Standard stored at 2-8° C. |
| L- Histidine 25 mM | 53.8 |
| L- Arginine 75 mM | |
| Sodium chloride 101 mM | |
| Polysorbate 80 0.02% w/v | |

TABLE 15

| | % Potency compared to Reference |
|---|---|
| Ingredient (QTY) | Standard stored at 2-8° C. |
| L- Histidine 25 mM | 70.8 |
| L- Arginine 75 mM | |
| Sodium chloride 101 mM | |
| Polysorbate 80 0.02% w/v | |
| Sucrose 0.1% w/v | |

TABLE 16

| | % Potency compared to Reference |
|---|---|
| Ingredient (QTY) | Standard stored at 2-8° C. |
| L- Histidine 25 mM | 65.7 |
| L- Arginine 75 mM | |
| Sodium chloride 101 mM | |
| Polysorbate 80 0.02% w/v | |
| Sucrose 0.2% w/v | |

TABLE 17

| | % Potency compared to Reference |
|---|---|
| Ingredient (QTY) | Standard stored at 2-8° C. |
| L- Histidine 25 mM | 81.6 |
| L- Arginine 75 mM | |
| Sodium chloride 101 mM | |
| Polysorbate 80 0.02% w/v | |
| Sucrose 0.5% w/v | |

Reference Standard Formulation Composition:

| Ingredient | (QTY) | |
|---|---|---|
| L- Histidine | 25 | mM |
| L- Arginine | 75 | mM |
| Sodium chloride | 101 | mM |
| Polysorbate 80 | 0.02% | w/v |
| Sucrose | 0.5% | w/v |

Conclusion: Highest stability was observed formulation comprising 0.5% Sucrose.

### Example 6

Analytical test for purity, stability, efficacy and potency of Dengue (VIS513) Mab formulation with storage at
2-8° C. for a period of 0 months, 3 months, 6 months, and 9 months.
25° C. for a period of 0 days and 30 days
40° C. for a period of 0 days, 7 days, 14 days, 28 days, 35 days, and 42 days.

6.1: Potency of VIS513 Antibody Formulation was Tested by Indirect ELISA.

The indirect ELISA based method was used to quantify binding of Dengue Mab (VIS513) to EDIII protein of DV1 antigen. EDIII protein was immobilized to the plate. Unbound antigen was removed by washing. In the next, step standard and test samples were added, allowed to bind to the antigen. To determine the amount of bound Dv-Mab, Mouse anti-Human IgG Fc-HRP, specific to Dv-Mab (human Immunoglobulin Fc fragment), was used to recognize the presence of Dv-Mab. The assay was developed with TMB Microwell Peroxidase Substrate System which quantifies the extent of binding by amount of color formed at 450 nm. The data analysis software generated a binding curve for each sample using a four parameter curve fitting model, and compared the binding curve of the test sample to the standard curve by calculating Relative Potency. The potency of a test sample is reported as % Activity relative to reference standard (Relative Potency times 100).

Results:

TABLE 18
Dengue (VIS513) Mab potency (%) by indirect
ELISA for formulation stored at 2-8° C.

| | 0 day | 3 months | 6 months | 9 months |
|---|---|---|---|---|
| Batch 1 | 74.90 | 79.30 | 89.30 | 84.9 |
| Batch 2 | 74.30 | 80.05 | 82.20 | 86.7 |

TABLE 19
Dengue (VIS513) Mab potency (%) by indirect
ELISA for formulation stored at 25° C.

| | 0 day | 30 days |
|---|---|---|
| Batch 1 | 74.90 | 79.30 |
| Batch 2 | 74.30 | 87.8 |

TABLE 20
Dengue (VIS513) Mab potency (%) by indirect
ELISA for formulation stored at 40° C.

| | 0 day | 14 days | 21 days | 28 days | 35 days | 42 days |
|---|---|---|---|---|---|---|
| Batch 1 | 70.3 | 82.80 | 96.20 | 71.90 | 89.70 | 90.80 |

Dengue (VIS513) mab formulation did not show any time dependent loss of binding affinity.

6.2: PRNT Assay to Determine EC50

The assay involves premixing serially diluted antibody with virus to allow antibody binding, neutralization then transfer of mixture to a Vero cell monolayer, overlay with a viscous medium, incubation (˜3-7 days, depending on virus serotype) to allow limited virus replication and spread, followed by detection of plaques. Neutralization was captured by the reduction of plaque formation. Robust detection was achieved with immunostaining methods, using mouse 4G2 Anti-Dengue antibody and HRP-labelled goat anti-mouse antibody with Peroxidase substrate.

The Dengue (VIS513) Mab formulation samples were been tested against all four serotypes of dengue viruses i.e. DV1, DV2, DV3 and DV4. EC50 value was calculated for neutralization of Dengue viruses. EC50 value represents the 50% effective concentration required for the effective neutralization of dengue viruses and EC50 value calculated from number of plaques present in the virus control wells and number of plaques in the wells in which mab-Virus incubated samples were added.

Results:

TABLE 21
Dengue (VIS513) mAb EC50 (ng/ml) value by PRNT
assay for formulation stored at 2-8° C.

| | Dengue | | | | |
|---|---|---|---|---|---|
| | virus | | | | |
| Batch # | serotypes | 0 day | 3 months | 6 months | 9 months |
| Batch 1 | DV1 | 47.15 | 49.74 | 23.88 | 24.12 |
| | DV2 | 5.9 | 8.03 | 3.58 | 3.31 |
| | DV3 | 14.38 | 14.7111 | 5.58 | 4.57 |
| | DV4 | 30.29 | 50.75 | 23.96 | 23.35 |
| Batch 2 | DV1 | 21.03 | 21.01 | 16.12 | 29.41 |
| | DV2 | 3.26 | 5.69 | 4.15 | 2.87 |
| | DV3 | 13.19 | 24.14 | 6.53 | 7.05 |
| | DV4 | 22.46 | 22.44 | 16.61 | 19.56 |

TABLE 22
Dengue (VIS513) Mab EC50 (ng/ml) value by
PRNT assay for formulation stored at 25° C.

| | Dengue | | |
|---|---|---|---|
| | virus | | |
| Batch # | serotypes | 0 day | 30 days |
| Batch 1 | DV1 | 47.15 | 29.82 |
| | DV2 | 5.9 | 5.6 |
| | DV3 | 14.38 | 9.63 |
| | DV4 | 30.29 | 35.28 |
| Batch 2 | DV1 | 21.03 | 28.34 |
| | DV2 | 3.26 | 5.51 |
| | DV3 | 13.19 | 9.25 |
| | DV4 | 22.46 | 30.69 |

TABLE 23
Dengue (VIS513) Mab EC50 (ng/ml) value by
PRNT assay for formulation stored at 40° C.

| | Dengue | | | |
|---|---|---|---|---|
| | virus DV2 | | | |
| Batch # | serotypes | | Control | Test |
| Batch 1 | 0 | day | 6.66 | — |
| | 7 | days | 6.37 | 26.89 |
| | 14 | days | 6.18 | 36.12 |

Dengue (VIS513) mab formulation did not show any time dependent loss of virus neutralization efficacy at 2-8° C. & 25° C. VIS513 formulation even if kept at 40° C., does not lose its ability to neutralize dengue virus.

6.3: Aggregation and Purity Analysis

A HPLC-based size exclusion chromatography (HPLC-SEC) was used to assess the aggregates in the bulk and final formulation of DV Mab. In this method a phenomenex Bio-Sec-S 3000 column was used to demonstrate the aggregates and monomer percentage of Dengue (VIS513) Mab by injecting the ˜50 ug of total antibody and run at a flow rate of 1 ml/minute for 35 minutes. Phosphate buffered Saline (PBS), pH 6.5 was used as mobile phase.

Results: SEC-HPLC (Acceptance Range is NLT 90%)

TABLE 24
SEC-HPLC analysis of formulation stored at 2-8° C.

| | 0 day | 3 months | 6 months | 9 months |
|---|---|---|---|---|
| Batch 1 | 99.21 | 98.60 | 99.14 | 98.55 |
| Batch 2 | 99.26 | 98.81 | 98.89 | 98.53 |

TABLE 25
SEC-HPLC analysis of formulation stored at 25° C.

| | 0 day | 30 days |
|---|---|---|
| Batch 1 | 99.21 | 97.63 |
| Batch 2 | 99.26 | 97.58 |

TABLE 26
SEC-HPLC analysis of formulation stored at 40° C.

| | 0 day | 7 days | 14 days | 21 days | 28 days | 35 days | 42 days |
|---|---|---|---|---|---|---|---|
| Batch 1 | 99.21 | 99.30 | 99.50 | 97.74 | 97.30 | 98.70 | 95.30 |

Dengue (VIS513) mab formulation did not show any significant time dependent aggregation; and purity/monomer content was found to be >98%.

Dengue (VIS513) mab formulation did not show any significant time dependent aggregation; and purity/monomer content was found to be >98%.

### Example 7

Effect of Surfactant Concentrations of Formulations:

Effect of Surfactant concentration was evaluated by sub-visible particle analysis. Formulations varying Polysorbate-80 strengths were prepared and analyzed for Sub visible particle analysis.

TABLE 27

| | Formulation with Polysorbate 80 conc (% W/V) | | |
|---|---|---|---|
| Particle size | 0.0016% | 0.002% | 0.005% |
| ≥2μ | 916 | 211 | 20 |
| ≥5μ | 55 | 48 | 5 |
| ≥10μ | 6 | 16 | 2 |
| ≥25μ | 1 | 1 | 0 |

Conclusion:

In the formulation containing 0.005% w/v Polysorbate-80 minimum sub visible particles were observed. Depending on dose, if the formulation requires dilution Polysorbate 80 strength was finalized 0.02% wN with margin of 4 fold.

### Example 8: Study of Determination of Minimum Concentration of Stabilizers Used

Minimum buffer strength required (10-30 mM) was referred from the available literature. To find out minimum Arginine (used as solubilising agent and viscosity reducing agent) Mab sample was buffer exchanged into normal saline and Arginine stock solution (300 mM) was gradually added. The aggregation of the solution was monitored by measuring OD@350 nm. The saline with 75 mM Arginine gave lowest OD hence 75 mM Arginine was finalized.

### Example 8

Viscosity Studies of Dengue (VIS513) Antibody Formulation

Viscosity of DV mab samples was measured on a microchip based Viscometer, Model: microVISC™ (Make: RheoSense, CA USA) as per procedure mentioned in the instrument manual.

TABLE 28

| Sample | Measurement Temperature | Viscosity mPa-S |
|---|---|---|
| Day 0 | 5° C. | 2.04 |
| | 25° C. | 1.164 |
| Day 90 stored at 2-8° C. | 25° C. | 1.173 |
| Day 30 stored at 25° C. | 25° C. | 1.137 |

Conclusion:

No time dependent increase in viscosity was observed in the mab formulation stored at 2-8° C. for 90 days as well at a sample kept at 25° C. for 1 month, this is primarily due to the excipient-Arginine 75 mM. Viscosity of our formulation was found to be 1.1 to 1.2 mPa-S/cP, which is lower than other marketed formulations that have viscosity between 11-50 mPa-S/cP

### Example 9: Viral Spiking Studies on Dengue (VIS513) mAb Purification Process

Virus validation was performed for actual manufacturing process, to test the effectiveness of the virus removal by virus filtration in the manufacturing process of monoclonal antibody.

Murine Leukemia Virus (MuLV) and Minute virus of mice (MMV/MVM) were used as model organisms. Inventors of this invention compared the ability of their inventive purification process with that of the general and well established method of monoclonal antibody purification.

The general and well established method of monoclonal antibody purification comprised of Protein-A Affinity Chromatography (GE Resin); Low pH Treatment; Sartobind Q Chromatography (Anion Exchange Membrane, Sartorius, single use); Sartobind Phenyl Chromatography (Membrane Chromatography, Sartorius, single use); Viresolve Pro filtration (Nanofiltration, Merck).

TABLE 29

Log₁₀ Viral Reduction Factor (LRV)

| Step | MuLV | MMV |
|---|---|---|
| General/Standard method | 12.64 ± 0.60 | 7.02 ± 0.69 |
| Inventive method (SIIPL) | 23.74 ± 0.60 | 11.91 ± 0.70 |

Results:

SIIPL purification process was highly efficient in viral clearance, total LRV achieved is as per the ICH guidelines. (Standard Process LRV 12.64 while SIIPL inventive process 23.74) Dengue antibody purified using our inventive process was found to be suitable for human clinical trials without any viral risk.

### Example 10: Upstream Process for Cell Culturing and Expression of Therapeutic Protein i.e. Rabies Monoclonal Antibody

Protocol:

Cell culturing at 2 L scale was carried out in a Fed batch manner using below mentioned parameters during fermentation/upstream process.
The rabies monoclonal antibody was expressed in cell line “GS-CHO”.
Cell culture medium used for cell growth and expression of therapeutic protein i.e. Rabies monoclonal antibody was “1× Cellvento™ CHO-220 Liquid Medium” or “Actipro 1× (Hyclone)”
Feed solution A, Feed solution B, Feed solution C, Feed solution D selected from group comprising of Glucose, Cell Boost™ 5 Supplement (Hyclone), EX-CELL 293 (Sigma Aldrich), Cell Boost 7a and 7b supplements (Hyclone), 3× Actipro (Hyclone), Cell Vento 220 (1× medium), EX-CELL® Advanced™ CHO Feed was used for supplementation feed.
pH of the fermentation medium was maintained at 6.5 to 7.5.
Osmolality of the fermentation medium was maintained between 270-450 mOsm/Kg.
Dissolved oxygen of the fermentation medium was maintained at about 20% to about 60%.
Temperature of the fermentation medium was maintained at 36.5±1.

Feed supplementation was done in a gradual drip manner as per following table:

TABLE 30

| | | | | | Basal | Basal |
|---|---|---|---|---|---|---|
| | | | | | Medium 1 | Medium 2 |
| | Feed | Feed | Feed | Feed | (3X Actipro″ | (Cellvento ™ |
| Day | A | B | C | D | (Hyclone) | CHO-220 (1X)) |
| 0 | | | | | | |
| 1 | | | | | | |
| 2 | | 4% | | | | |
| 3 | 0.2% | | | | | |
| 4 | | | 4% | 0.4% | 10% | 8% |
| 5 | | 6% | 4% | 0.4% | | |
| 6 | 0.2% | 4% | 4% | 0.4% | | |
| 7 | | 6% | | | | |
| 8 | 0.2% | | 4% | 0.4% | | |
| 9 | | 4% | 4% | 4.0% | | |
| 10 | 0.1% | 2% | 4% | 0.4% | | |
| 11 | 0.2% | 2% | 2% | 0.2% | | |
| 12 | | 4% | | | | |
| 13 | 0.1% | 2% | 2% | 0.2% | | |
| 14 | | 3% | | | | |
| 15 | | 3% | | | | |
| 16 | | | | | | |

Note:
All feeding solutions may vary by ±1% and by ±1 day.

The cell culture was harvested upon drop in OD up to 60%

Results & Conclusion:

Yield of 3-5 gm/L was obtained of the fermentation process. Harvest obtained was further subjected to purification/downstream processing.

### Example 11

Cell culture obtained according to example 9 was harvested and later subjected to protocol for purification of the rabies monoclonal antibody as per FIG. 1.

Detailed process used was as follows:
Protein-A Affinity Chromatography:
   In this step targeted monoclonal antibody was separated from the media components in the harvested supernatant. Clarified supernatant was passed through the chromatography column and then eluted using compatible elution buffers.
   Materials used:
Resin (Matrix): Mab Select Sure/Eshmuno A (Protein-A Affinity)
Residence Time: 4.0-8.0 minutes
Column used: XK 26
Equilibration Buffer: 20 mM Phosphate Buffer+150 mM NaCl+0.05% (w/v) Polysorbate 80, pH 7.0±0.2.
Wash I Buffer: 20 mM Phosphate Buffer+150 mM NaCl+0.05% (w/v) Polysorbate 80, pH 7.0±0.2.
Wash II Buffer: 20 mM Phosphate Buffer+1M NaCl+0.05% (w/v) Polysorbate 80, pH 7.0±0.2.
Wash III Buffer: 10 mM Phosphate Buffer+125 mM NaCl+0.025% (w/v) Polysorbate 80, pH 6.0±0.2.
Elution Buffer: 20 mM Citrate buffer+0.025% (w/v) Polysorbate 80, pH 3.0±0.2.
CIP Buffer: 0.1 M NaOH

Process Parameters Used:

TABLE 31

| | | | Linear Flow Rate |
|---|---|---|---|
| S. No. | Process Step | Column Volume | (cm/hr) |
| 1 | Equilibration | 5 | <300 |
| 2 | Loading (mL) | As Actual | <300 |
| 3 | Wash I | 2 | <300 |
| 4 | Wash II | 4 | <300 |
| 5 | Wash III | 4 | <300 |
| 6 | Elution | 5 | <300 |
| 7 | Cleaning | 3 | <300 |
| 8 | Storage | 2 | <300 |

1. Low pH Viral Inactivation
i. Eluate from protein-A Affinity chromatography was subjected to low pH i.e. 3.5±0.1 for 60±10 minutes to inactivate the viral particles.
ii. After low pH hold, eluate was neutralized using neutralization buffer i.e. 1 M Tris/Citrate buffer having pH 7.0±0.2. Subsequently neutralized solution was filtered using 0.8/0.45μ or 0.8/0.2μ.
iii. Conductivity of neutralized eluate was adjusted using WFI with 0.025% (w/v) Polysorbate 80.

2. Cation Exchange Chromatography

Positively charged antibody molecules bound with the column while negatively charged molecules come in the flow through. Column bound antibody molecules are eluted using salt gradient.

Materials Used:

Resin used: Fractogel SO3⁻/Fractogel SE Hicap (Merck)

Residence Time: 4.00-7.00 minutes

Column used: XK 26

Pre Equilibration: 200 mM Citrate buffer pH 6.0±0.2.

Equilibration: 10 mM Citrate buffer+0.025% (w/v) Polysorbate 80, pH 6.0±0.2.

Loading: Low pH Hold neutralized.

Wash Buffer A: 10 mM Citrate buffer, pH 6.0±0.2.

Wash Buffer B: 20 mM Citrate buffer+300 mM NaCl, pH 6.0±0.2.

CIP Buffer: 0.5 M NaOH

Storage Buffer: 20% ethanol+150 mM NaCl

TABLE 32
Process Parameters:

| | | | Linear Flow Rate |
|---|---|---|---|
| S. No. | Process Step | CV | (cm/hr) |
| 1 | Pre Equilibration | 2 | <300 |
| 2 | Equilibration | 5 | <300 |
| 3 | Loading | As Actual | <300 |
| 4 | Wash | 5 | <300 |
| 5 | Elution 0-60% B (Gradient) | 15 | <300 |
| 6 | 100% B | 2 | <300 |
| 7 | CIP | 3 | <300 |

TABLE 33
Fraction collection during gradient

| | | Collection Criteria (UV₂₈₀) |
|---|---|---|
| S. No. | Fraction Name | mAU |
| 1 | Fraction 01 | Upto 300 |
| 2 | Fraction 02 | 300-700 |
| 3 | Fraction 03 | 700 till base line |
| 4 | Fraction 04 (100% B) | Baseline to base line |

Anion Exchange Chromatography:

All negatively charged impurities are bound with the membrane while antibody comes in the flow through.

Materials Used:

Membrane/Resin used: Sartobind Q single Sep mini (Sartorius)/Eshmuno Q

Loading volume: 150 mg/mL-1000 mg/mL

Column used: XK 26

Cleaning Buffer: 0.5 M NaOH

Pre-equilibration buffer: 200 mM Citrate buffer, pH 6.0±0.2.

Equilibration Buffer: 20 mM Citrate buffer pH 6.0±0.2; and optionally 0.025% PS-80 pH 6.0±0.2

Storage Buffer: 20% ethanol+150 mM NaCl or 0.1 M NaOH

3. Nano

TABLE 34
Process Parameters:

| | | Column Volume | Linear Flow Rate |
|---|---|---|---|
| S. No. | Process Step | (CV) | (cm/hr) |
| 1 | Cleaning | 10 | <300 |
| 2 | Pre Equilibration | 10 | <300 |
| 3 | Equilibration | 20 | <300 |
| 4 | Loading | As Actual | <300 |
| 5 | Post Load Wash | 20 | <300 |
| 6 | Cleaning | 10 | <300 |

filtration:

20 nm nanofilter i.e. Viresolve PRO (Merck), was used to remove any virus particles available in the therapeutic protein.

4. Tangential Flow Filtration/Ultra Flow Filtration:

The antibody was concentrated to desired concentration and buffer exchanged in one of the three formulation buffers.

Material Used:

Formulation Buffer:
Buffer 1: 25 mM Histidine buffer+75 mM Arginine Buffer+75 mM NaCl, pH 6.50±0.25;
Buffer 2: 25 mM Histidine, 75 mM Arginine, 101 mM NaCl;
Buffer 3: 25 mM Histidine, 75 mM Arginine, 75 mM to 101 mM NaCl, Polysorbate-80 0.002% w/v
Cleaning Buffer: 0.5 M NaOH

Storage Buffer: 20% ethanol+150 mM NaCl or 0.1 M NaOH

Membrane used: PALL Centramate T Series, PES membrane MWCO: 30 kDa

TABLE 35
Process Parameters:

| S. No. | Process Step | Description | Remark |
|---|---|---|---|
| 1 | Cleaning | 0.5M NaOH | 30 Minutes |
| | | | recirculation |
| 2 | WFI Cleaning | WFI, till conductivity | — |
| | | comes below 1.3 μS/cm | |
| 3 | Equilibration | 400 ml | — |
| 4 | Concentration and | ~10-12 DV pass | — |
| | Diafiltration | | |
| 5 | WFI | WFI wash 1000 mL | — |
| 6 | Cleaning | 0.5M NaOH | 30 Minutes |
| | | | recirculation |

Sterile Filtration

Stabilizer was added to the antibody solution and sterile filtered through 0.2μ filter.

Results:

TABLE 36
Stage wise recovery of the various steps
used in the purification process.

| S. No. | Stage | Recovery (%) * |
|---|---|---|
| 1 | Protein-A Affinity Chromatography | 94 |
| 2 | Low pH Viral Inactivation | 98 |
| 3 | Cation Exchange Chromatography | 94 |
| 4 | Anion Exchange Chromatography | 95 |
| 5 | Nanofiltration | 100 |
| 6 | TFF/UFF | 98 |
| 7 | Formulation and Sterile filtration | 100 |

The overall process recovery was found to be >80%.

TABLE 37
Impurity data

| Assay | Accepted Criterion | | Batch 1 | Batch 2 |
|---|---|---|---|---|
| Concentration by | — | | 26.19 | 24.39 |
| UV280 (mg/ml) | | | | |
| Purity by SEC-HPLC | Monomer should be >90.00%. | | 100 | 100 |
| (% monomer) | Retention time of monomer | | | |
| | should be comparable to | | | |
| | reference standard | | | |
| SDS PAGE NR (kD) | — | | 156.0 | 156.0 |
| SDS PAGE R (kD) | — | | 50.4/27.7 | 50.7/27.9 |
| Residual CHO DNA | <2 | pg/mg IgG | 0.34 | 0.17 |
| (pg/mg of mAb) | | | | |
| Residual Protein A | ≤10.00 | ng/mg IgG | <10 | <10 |
| (ng/mg of mAb) | | | | |
| Residual CHO Protein | ≤100.00 | ng/mg IgG | 5.50 | 7.94 |
| (ng/mg of mAb) | | | | |
| Bacterial Endotoxin | ≤0.1 | EU/mg of protein | 0.35 | 0.5 |
| (EU/mg of protein) | | | | |

TABLE 38
Batch wise Recovery & Purity

| S. No. | Batch No. | Recovery (%) | Purity (%) |
|---|---|---|---|
| 1 | Batch 1 | 82 | 99.4 |
| 2 | Batch 2 | 80 | 99.6 |

The overall purity of the rabies mab after purification was found to be >99% and overall recovery was found to be >80%.

### Example 12

Purified Rabies monoclonal antibody was formulated as per the flowchart given in FIG. 2.

Excipients i.e. Arginine, Histidine, NaCl, Sucrose, and polysorbate-80 were added and mixed thoroughly using a magnetic stirrer at 50-60 RPM to form a mixture of excipients. This mixture was then added into the Dengue mAb TFF harvest gradually with stirring rate 50-60 RPM. pH was checked (pH 6.5) and if required adjusted by histidine-arginine buffer. The final formulation was filtered through a 0.2 μM filter and filled into final container.

TABLE 39
The concentration of each component in the final formulation was as follows:

| Ingredient | Formulation 1 | | Formulation 2 | | Formulation 3 | |
|---|---|---|---|---|---|---|
| Rabies Mab | 10 | mg/ml | 25 | mg/ml | 50 | mg/ml |
| Histidine | 25 | mM | 25 | mM | 25 | mM |
| Arginine | 75 | mM | 75 | mM | 75 | mM |
| Sodium Chloride | 101 | mM | 101 | mM | 101 | mM |
| Sucrose | 0.5% | w/v | 0.5% | w/v | 0.5% | w/v |
| Polysorbate-80 | 0.02% | w/v | 0.02% | w/v | 0.02% | w/v |
| pH | 6.5 + 0.5 | | 6.5 + 0.5 | | 6.5 + 0.5 | |
| Osmolality | 386 | mOsm/kg | 386 | mOsm/kg | 386 | mOsm/kg |

These formulations were further tested for purity, stability, efficacy and potency for 9 months.

### Example 13: Analytical Test for Purity & Stability of Rabies Mab Formulation with Storage at 2-8, 25 and 40° C. for a Period of 0 Months, 1 Month, 3 Months, & 6 Months

13.1 Aggregation and Purity Analysis

A HPLC-based size exclusion chromatography (HPLC-SEC) was used to assess the aggregates in the bulk and final formulation of DV Mab. In this method a phenomenex Bio-Sec-S 3000 column was used to demonstrate the aggregates and monomer percentage of Rabies Mab by injecting the ˜50 ug of total antibody and run at a flow rate of 1 ml/minute for 35 minutes. Phosphate buffered Saline (PBS), pH 6.5 was used as mobile phase.

Results:

TABLE 40
SE- HPLC (%) results

| Temp. | 0 day | 1 months | 3 months | 6 months |
|---|---|---|---|---|
| 2-8° C. | 100 | 99.70 | 99.40 | 99.60 |
| 25° C. | 100 | 99.60 | — | — |
| 40° C. | 100 | 99.20 (7 days) | — | — |

Rabies mab formulation did not show any time dependent aggregation and purity/monomer content was found to be >99%.

13.2 SDS Page Analysis Batch 1—Test Sample at 2-8° C.

TABLE 41

| | | | 0 Day | 1 M | 3 M | 6 M |
|---|---|---|---|---|---|---|
| SDS PAGE | Test | Heavy Chain | 50.8 | 49.8 | 49.3 | 49.2 |
| REDUCING (kD) | Sample at | Light Chain | 25.6 | 25.7 | 25.6 | 26.4 |
| Molecular | 2-8° C. | Heavy Chain | 50 | 50 | 50 | 50 |
| weight of heavy | Reference | Light Chain | 25 | 25 | 25 | 25 |
| and light chains | standard | | | | | |
| must be within ±10% | | | | | | |
| of molecular weight of | | | | | | |
| reference standard. | | | | | | |
| Total % of heavy and | | | | | | |
| light chain must be >95%. | | | | | | |
| Total %: 100 | | | | | | |

| | | | | | |
|---|---|---|---|---|---|
| SDS-PAGE | Test Sample at | 151.0 | 156.7 | 157.2 | 157.6 |
| Non-Reducing (kD) | 2-8° C. | | | | |
| RS: Reference | Reference standard | 150 | 150 | 150 | 150 |
| standard | | | | | |
| Molecular weight | | | | | |
| of major band must | | | | | |
| be within ±10% of | | | | | |
| molecular weight of | | | | | |
| reference standard (RS). | | | | | |

Test sample at 25° C.

| | | | 0 Day | 30 Days |
|---|---|---|---|---|
| SDS PAGE | Test | Heavy Chain | 50.8 | 47.5 |
| REDUCING (kD) | Sample at | Light Chain | 25.6 | 25.5 |
| Molecular | 25° C. | Heavy Chain | 50 | 50 |
| weight of heavy | Reference | Light Chain | 25 | 25 |
| and light chains | standard | | | |
| must be within ±10% | | | | |
| of molecular weight of | | | | |
| reference standard. | | | | |
| Total % of heavy and | | | | |
| light chain must be >95%. | | | | |
| Total %: 100 | | | | |

| | | | |
|---|---|---|---|
| SDS-PAGE | Test Sample at | 151.0 | 151.6 |
| Non-Reducing (kD) | 25° C. | | |
| RS: Reference | Reference standard | 150 | 150 |
| standard | | | |
| Molecular weight | | | |
| of major band must | | | |
| be within ±10% of | | | |
| molecular weight of | | | |
| reference standard (RS). | | | |

Conclusion: Rabies mAb formulation did not show any significant time dependent changes in aggregation and change in molecular weight. Hence the formulation is found to be stable at 2-8° C. 25° C., AND 40° C.

```
Sequence_ST25.txt
Seq ID 1: VH amino acid sequence of VIS513 (Dengue
Monoclonal Antibody)
QVQLVQSGAEVKKPGASVKVSCKAGFNIKDVYMSWVRQAPEQGLEWMGRIDPENGDTKYD
  60
PKLQGRVTMTADTSTNTAYMELRSLRSDDTAVYYCARGWEGFAYWGQGTLVTVSSASTKG
120
PSVFPLAPSSKSTSGGTAALGCLVKDYFPEPVTVSWNSGALTSGVHTFPAVLQSSGLYSL
180
SSVVTVPSSSLGTQTYICNVNHKPSNTKVDKRVEPKSCDKTHTCPPCPAPELLGGPSVFL
240
FPPKPKDTLMISRTPEVTCVVVDVSHEDPEVKFNWYVDGVEVHNAKTKPREEQYNSTYRV
300
VSVLTVLHQDWLNGKEYKCKVSNKALPAPIEKTISKAKGQPREPQVYTLPPSREEMTKNQ
360
VSLTCLVKGFYPSDIAVEWESNGQPENNYKTTPPVLDSDGSFFLYSKLTVDKSRWQQGNV
420
FSCSVMHEALHNHYTQKSLSLSPGK
445
Seq ID 2: VL amino acid sequence of VIS513 (Dengue
Monoclonal Antibody)
DIVMTQSPASLAVSLGERATISCRASENVDKYGNSFMHWYQQKPGQPPKLLIYRASELQW
  60
GVPDRFSGSGSGTDFTLTISSLQAEDVAVYYCQRSNEVPWTFGQGTKLEIKRTVAAPSVF
120
IFPPSDEQLKSGTASVVCLLNNFYPREAKVQWKVDNALQSGNSQESVTEQDSKDSTYSLS
180
STLTLSKADYEKHKVYACEVTHQGLSSPVTKSFNRGEC
218
Seq ID 3: SII RmAb (RAB1)(17C7) Heavy Chain Amino Acid
Sequence
QVQLVESGGGVVQPGRSLRLSCAASGFTFSTYAMHWVRQAPGKGLEWVAVVSYDGRTKDY
  60
ADSVKGRFTISRDNSKNTLYLQMNSLRTEDTAVYFCARERFSGAYFDYWGQGTLVTVSSA
120
STKGPSVFPLAPSSKSTSGGTAALGCLVKDYFPEPVTVSWNSGALTSGVHTFPAVLQSSG
180
LYSLSSVVTVPSSSLGTQTYICNVNHKPSNTKVDKRVEPKSCDKTHTCPPCPAPELLGGP
240
SVFLFPPKPKDTLMISRTPEVTCVVVDVSHEDPEVKFNWYVDGVEVHNAKTKPREEQYNS
300
TYRVVSVLTVLHQDWLNGKEYKCKVSNKALPAPIEKTISKAKGQPREPQVYTLPPSREEM
360
TKNQVSLTCLVKGFYPSDIAVEWESNGQPENNYKTTPPVLDSDGSFFLYSKLTVDKSRWQ
420
QGNVFSCSVMHEALHNHYTQKSLSLSPGK
449
Seq ID 4: SII RMAb (RAB1)(17C7) Light Chain Amino Acid
Sequence
EIVLTQSPATLSLSPGERATLSCRASQSVSSYLAWYQQKPGQAPRLLIYDASNRATGIPA
  60
RFSGSGSGTDFTLTISSLEPEDFAVYSCQQRNNWPPTFGGGTKVEIKRTVAAPSVSVFIF
120
PPSDEQLKSGTASVVCLLNNFYPREAKVQWKVDNALQSGNSQESVTEQDSKDSTYSLSST
180
LTLSKADYEKHKVYACEVTHQGLSSPVTKSFNRGEC
219
```

We claim:

1. A method of manufacturing a pharmaceutical antigen binding protein with high yield and minimum aggregation, comprising:
a) culturing large scale mammalian cells that express antigen binding protein in a cell culture production medium, wherein the method effectively maintains the cell count and results in a yield of least 2 gm/L;
b) purification of antigen binding protein from harvested supernatant obtained in step (a), wherein the method results in recovery of at least 80% and purity of at last 99%; and
c) obtaining a stable antigen binding protein formulation, wherein Osmolality of the stable formulation is in a range of 300-400 mOsm/Kg and viscosity of the stable formulation is less than 2.5 mPa-S.

2. The method of claim 1 comprising culturing large scale mammalian cells that express antibody in a cell culture production medium; wherein the culturing step includes use of a basal medium, use of a concentrated basal medium as a feed solution, and use of feed solutions along-with a definite feeding strategy, resulting in enhanced cell growth, maintaining lower concentrations of lactate and ammonia, and effectively maintaining the cell count thereby increasing cell longevity and high yield.

3. The method of claim 2, wherein the cell culture production medium comprises of at least one medium selected from the group comprising Cell Vento 220 (Merck), ACTIPRO (HyClone/GE), and Gibco™ Dynamism™ Medium (Thermo Fisher).

4. The method of claim 1, wherein the cell culture production medium is supplemented with one or more other nutrients, at least once during the method.

5. The method of claim 1, wherein the cell culture production medium is supplemented on a schedule comprising supplementation that is continuous, daily, every other day, every two days, or any combination thereof.

6. The method of claim 4, wherein the cell culture production medium is supplemented with a feed solution comprising at least one medium selected from the group comprising Glucose, Cell Boost™ 5 Supplement (Hyclone), EX-CELL 293 (Sigma Aldrich), Cell Boost 7a and 7b supplements (Hyclone), 3× Actipro medium, Cell Vento 220 (3× medium), EX-CELLO Advanced™ CHO Feed 1, EfficientFeed™ A, EfficientFeed™ B, and EfficientFeed™ C, and any combination thereof.

7. The method of claim 1; wherein the cell count is in the range of 10×10⁶-20×10⁶ cells/ml.

8. The method of claim 1, wherein the cell culture production medium has an Osmolality in a range of 250-500 mOsm/Kg; a pH in a range of 6.5-7.5; dissolved oxygen is maintained in a range of 10-60%; Cell culture temperature is in a range of 30% C to 38° C.; Glucose concentration is maintained below 7%; harvesting the cell culture when viability is decreased to 80%; wherein cell culture conditions are maintained in a manner such that Lactate concentration is not more than 5 g/L; and Ammonia concentration is not more than 5 mMol/L.

9.-10. (canceled)

11. The method of claim 1, wherein the antigen binding protein is selected from the group comprising of humanized antibody, chimeric antibody, human antibody, bispecific antibody, multivalent antibody, multi-specific antibody, antigen binding protein fragments, polyclonal, monoclonal, diabodies, nanobodies, monovalent, bispecific, heteroconjugate, multispecific, autoantibodies, single chain antibodies, Fab fragments, F(ab)′2, fragments, fragments produced by a Fab expression library, anti-idiotypic (anti-Id) antibodies, epitope-binding fragments and CDR-containing fragments and combinations thereof.

12. The method of claim 1, wherein the cell culture Production medium comprises a cell line selected from the group consisting of Chinese Hamster Ovary (CHO) cells, GS-CHO, CHOK1SV GS-KO, CHO DUX-B11, CHO-K1, BSC-1, NSO myeloma cells, CV-1 in Origin carrying SV40 (COS) cells, COS-1, COS-7, P3X3Ag8.653, SP2 cells, human embryonic kidney (HEK 293) cells, baby hamster kidney (BHK 21) cells, African green monkey kidney VERO-76 cells, HELA cells, human lung cells (W138), Retinal cells, and human hepatoma line (Hep G2). VERO, BHK, MDCK, W138 cells, NIH-3T3, W138, BT483, Hs578T, HTB2, BT20, T47D, NS0 (a murine myeloma cell line that does not endogenously produce any immunoglobulin chains), CRL7O3O, HsS78Bst cells, PER.C6, SP2/0-Ag14, a myeloma cell line, a hybridoma cell line, human lung cells (W138), Retinal cells, human hepatoma line (Hep G2), CHO-K1 (ATCC CCL-61), DGI 44, SH87 cellCHO-DXB11, CHO designated ECACC 85050302, CHO clone 13 (GEIMG, Genova, IT), CHO clone B (GEIMG, Genova, IT), CHO-K1/SF designated ECACC 93061607 (CAMR, Salisbury, Wiltshire, UK), RR-CHOK1 designated ECACC 92052129 (CAMR, Salisbury, Wiltshire, UK), CHOK1sv, CHO-S, dihydrofolate reductase negative CHO cells (CHO/−DHFR), and dp12.CHO cells; monkey kidney CV1 cells transformed by SV40 (COS cells, COS-7, ATCC CRL-1651); human embryonic kidney cells; baby hamster kidney cells (BHK, ATCC CCL-10); CAP cell, AGE1.HN cell, monkey kidney cells (CV 1, ATCC CCL-70); African green monkey kidney cells (VERO-76, ATCC CRL-1587; VERO, ATCC CCL-81); mouse sertoli cells (TM4); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TR1 cells; MCR 5 cells; and FS4 cells and hybridoma cells.

13. The method of claim 12, wherein the cell line is CHO-K1 SV GS-KO.

14. The method of claim 12, wherein the cell line is GS-CHO.

15. The method of claim 1, the cells are cultivated in a batch, fed batch-, continuous mode, or perfusion mod.

16. The method of claim 1, wherein the salt concentration of the buffers used in purification is in the range of 30 mM-500 mM.

17. (canceled)

18. The method of claim 1, wherein the purification step comprises one or more of Protein A affinity chromatography, cation exchange chromatography, and anion exchange chromatography.

19. The method of claim 1, wherein the purification step comprises affinity chromatography, Low pH viral inactivation, cation exchange chromatography, anion exchange chromatography, nanofiltration, and Tangential flow filtration/Ultrafiltration in a sequential manner.

20. The method of claim 1, wherein the purification step comprises affinity chromatography, Low pH viral inactivation, anion exchange chromatography, cation exchange chromatography, nanofiltration, and Tangential flow filtration/Ultrafiltration in a sequential manner.

21. The method of claim 1, wherein the purification step occurs in an affinity chromatography matrix selected from Protein A, Protein G and Protein L.

22. The method of claim 1, further comprising an additional chromatography step selected from the group comprising one or more of Hydrophobic interaction chromatography, Hydrophobic charge induction chromatography, Ceramic hydroxyapatite chromatography, Multimodal chromatography (Capto MMC and Capto Adhere), Membrane chromatography (Q membranes including Intercept™ (Millipore), Mustang® (Pall Corporation) and Sartobind™ (Sartorius)).

23. The method of claim 21, wherein the protein A chromatography matrix comprises one or more resins selected from the group comprising Eshmuno A, KanCapA™, MabSelect SuRe™, MabSelect SuRe LX, MabSelect Xtra, rProtein A Sepharose Fast Flow, Poros® MabCapture A, Amsphere™ Protein A JWT203, ProSep HC, ProSep Ultra, and ProSep Ultra Plus.

24. (canceled)

25. The method of claim 18, wherein viral inactivation of an eluate from the protein A affinity chromatography is accomplished by holding the eluate at a pH of 3.3-3.5 for a period of 50-100 minutes.

26. The method of claim 18, wherein the cation exchange chromatography is conducted using a resin selected from the group comprising one or more of sulfonate based group (e.g., MonoS, MiniS, Source 15S and 30S, SP SEPHAROSE® Fast Flow, SP SEPHAROSE® High Performance from GE Healthcare, TOYOPEARL® SP-650S and SP-650M from Tosoh, MACRO-PREP® High S from BioRad, Ceramic HyperD S, TRISACRYL® M and LS SP and Spherodex LS SP from Pall Technologies); a sulfoethyl based group (e.g., FRACTOGEL® SE, from EMD, POROS® S-10 and S-20 from Applied Biosystems); a sulphopropyl based group (e.g., TSK Gel SP 5PW and SP-5PW-HR from Tosoh, POROS® HS-20, HS 50, and POROS® XS from Life Technologies); a sulfoisobutyl based group (e.g., FRACTOGEL® EMD S03 “from EMD); a sulfoxy ethyl based group (e.g., SE52, SE53 and Express-Ion S from Whatman), a carboxymethyl based group (e.g., CM SEPHAROSE® Fast Flow from GE Healthcare, Hydrocell CM from Biochrom Labs Inc., MACRO-PREP® CM from BioRad, Ceramic HyperD CM, TRISACRYL® M CM, TRISACRYL® LS CM, from Pall Technologies, Matrex CELLUFINE® C500 and C200 from Millipore, CM52, CM32, CM23 and Express-Ion C from Whatman, TOYOPEARL® CM-650S, CM-650M and CM-650C from Tosoh); sulfonic and carboxylic acid based groups (e.g., BAKERBOND® Carboxy-Sulfon from J.T. Baker); a carboxylic acid based group (e.g., WP CBX from J.T Baker, DOWEX® MAC-3 from Dow Liquid Separations, AMBERLITE® Weak Cation Exchangers, DOWEX® Weak Cation Exchanger, and DIAION® Weak Cation Exchangers from Sigma-Aldrich and FRACTOGEL® EMD COO-from EMD); a sulfonic acid based group (e.g., Hydrocell SP from Biochrom Labs Inc., DOWEX® Fine Mesh Strong Acid Cation Resin from Dow Liquid Separations, UNOsphere S, WP Sulfonic from J.T. Baker, SARTOBIND® S membrane from Sartorius, AMBERLITE® Strong Cation Exchangers, DOWEX® Strong Cation and DIAION® Strong Cation Exchanger from Sigma-Aldrich); and a orthophosphate based group (e.g., PI 1 from Whatman).

27. (canceled)

28. The method of claim 18, the anion exchange chromatography is conducted using a resin selected from the group comprising one or more of DEAE cellulose, POROS® PI 20, PI 50, HQ 10, HQ 20, HQ 50, D 50 from Applied Biosystems, SARTOBIND® Q from Sartorius, MonoQ, MiniQ, Source 15Q and 30Q, Q, DEAE and ANX SEPHAROSE® Fast Flow, Q SEPHAROSE (GE), Q SEPHAROSE® High Performance, QAE SEPHADEX® and FAST Q SEPHAROSE® (GE Healthcare), WP PEI, WP DEAM, WP QUAT from J.T. Baker, Hydrocell DEAE and Hydrocell QA from Biochrom Labs Inc., U Osphere Q, MACRO-PREP DEAE and MACRO-PREP® High Q from Biorad, Ceramic HyperD Q, ceramic HyperD DEAE, TRISACRYL® M and LS DEAE, Spherodex LS DEAE, QMA SPHEROSIL® LS, QMA SPHEROSIL® M and MUSTANG® Q from Pall Technologies, DOWEX® Fine Mesh Strong Base Type I and Type II Anion Resins and DOWEX® MONOSPHER E 77, weak base anion from Dow Liquid Separations, INTERCEPT® Q membrane, Matrex CELLUFINE® A200, A500, Q500, and Q800, from Millipore, FRACTOGEL® EMD TMAE, FRACTOGEL® EMD DEAE and FRACTOGEL® EMD DMAE from EMD, AMBERLITE® weak strong anion exchangers type I and II, DOWEX® weak and strong anion exchangers type I and II, DIAION® weak and strong anion exchangers type I and II, DUOLITE® from Sigma-Aldrich, TSK gel Q and DEAE 5PW and 5PW-HR, TOYOPEARL® SuperQ-650S, 650M and 650C, QAE-550C and 650S, DEAE-650M and 650C from Tosoh, QA52, DE23, DE32, DE51, DE52, DE53, Express-Ion D and Express-Ion Q from Whatman.

29. (canceled)

30. The method of claim 18, wherein the anion exchange chromatography is in “flow through and wash mode” or “bind and elute mode.”

31. The method of claim 18, wherein removal of viral particles is accomplished by nanofiltration using a virus retentive filter selected from the group comprising one or more of Viresolve PRO (Merck), Planova 20N (Asahi Kasei), Bio EXL PALL PEGASUS PRIME, PEGASUS SV4 (Pall Life Sciences), and Virosart (Sartorius), Virosart CPV filter from Sartorius, Virosolve from Millipore, Ultipor DV20 or DV50 from Pall, Planova 20N and 50N, and BioEx from Asahi.

32. The method of claim 18, wherein the antigen binding protein is further concentrated using Tangential Flow Filtration (TFF).

33. The method of claim 32, wherein the TFF is carried out using a 30 kDa membrane selected from the group comprising one or more of Centramate T series PES membrane (Pall Corporation), Hydrosart (Sartorius), and Pelicon 3 (Merck).

34. (canceled)

35. The method of claim 1, wherein the stable antigen binding protein formulation contains no greater than 2% aggregates.

36. The method of claim 1, wherein the stable antigen binding protein formulation comprises of least one antigen binding protein, at least one Stabilizer, one Buffer, at least one Tonicity agent, and at least one surfactant.

37.-49. (canceled)

50. The method of claim 1, wherein the stable antigen binding protein formulation comprises about 1 mg/ml to about 100 mg/ml of the antigen binding protein.

51.-52. (canceled)

53. The method of claim 1, wherein the concentration of the antigen binding protein monomer in the stable antigen binding protein formulation is greater than 99%; residual CHO DNA is not more than 2 pg/mg of antigen binding protein, more particularly not more than 0.1 pg/mg of antigen binding protein; residual CHO protein is not more than 100 ng/mg of antigen binding protein, more particularly not more than 10 ng/mg of antigen binding protein; residual Protein-A is not more than 10 ng/mg of antigen binding protein, more particularly not more than 1.5 ng/mg of antigen binding protein; Endotoxin is not more than 0.1 EU/mg of antigen binding protein; viral clearance LRV for MuLV is atleast at least 20 fold 15 log 10, reduction factor and for MMV is atleast at least 12 log 10, reduction factor 10 fold.

54. A pharmaceutical formulation prepared according to the method of claim 1.

55. The pharmaceutical formulation of claim 54 comprising an antigen binding protein, a buffering agent, a tonicity agent, a surfactant and a stabilizing agent.

56. (canceled)

57.-68. (canceled)

69. A pharmaceutical formulation comprising
a. 1-100 mg/ml of at least one antigen binding protein;
b. 20-40 mM of Histidine;
c. 50-100 mM of Arginine;
d. 0.002-0.02% of Polysorbate 80 (w/v);
e. 50-150 mM NaCl; and
f. not more than 2.5% Sucrose w/v; wherein pH of the formulation is 6.5±0.5;
wherein the Osmolality of the formulation is 300-450 mOsmol/kg and viscosity is less than 2.5 mPa-S and said formulation is stable at 2-8 deg C. for at least 9 months, at 25 deg C. for at least 1 month, at 40 deg C. for at least 40 days, and at 50 deg C. for at least 2 days.

70. The pharmaceutical formulation of claim 69, wherein the formulation comprises 2-80 mg/ml of a least one antigen binding protein; 25 mM of Histidine; 75 mM of Arginine; 101 mM NaCl; 0.02% Polysorbate 80 (w/v); and 0.5% Sucrose w/v; wherein pH of the formulation is 6.5±0.5, and wherein the osmolality of the formulation is about 380 mOsmol/kg.

71.-74. (canceled)

75. The pharmaceutical formulation according to claim 69, wherein said antigen binding protein is a monoclonal antibody that binds to a dengue virus.

76. The pharmaceutical formulation according to claim 69, wherein said antigen binding protein is a monoclonal antibody that binds to a rabies virus.

77. The pharmaceutical formulation according to claim 70, wherein said antigen binding protein is a Dengue monoclonal antibody.

78. The pharmaceutical formulation according to claim 77, comprising 25 mg/ml of the Dengue monoclonal antibody.

79. The pharmaceutical formulation according to claim 77, comprising 50 mg/ml of the Dengue monoclonal antibody.

80. The pharmaceutical formulation according to claim 70, wherein said antigen binding protein is a Rabies monoclonal antibody.

81. The pharmaceutical formulation according to claim 80, comprising 25 mg/ml of the Rabies monoclonal antibody.

82. The pharmaceutical formulation according to claim 80, comprising 50 mg/ml of the Rabies monoclonal antibody.

83. The pharmaceutical formulation according to claim 70, wherein the formulation is a liquid formulation.

84. The pharmaceutical formulation according to claim 70, wherein the formulation is a lyophilized formulation.

85.-86. (canceled)

87. The pharmaceutical formulation according to claim 70, wherein said antigen binding protein is an anti-dengue antibody or an anti-rabies antibody that can be administered alone or in combination with other agents, other prophylactics or therapeutic modalities.

88.-89. (canceled)
